(19) United States
(12) Patent Application Publication
(10) Pub. No.: US 2023/0172944 A1
(43) Pub. Date: Jun. 8, 2023

(54) ROCK INHIBITORS FOR USE IN TREATING OR PREVENTING PULMONARY EDEMA

(71) Applicant: ATRIVA THERAPEUTICS GMBH, Tuebingen (DE)

(72) Inventors: Irina KUZNETSOVA,, Giessen (DE); Susanne HEROLD, Giessen (DE); John ZIEBUHR, Asslar (DE); Stephan PLESCHKA, Giessen (DE); Christin PETERANDERL, Echzell (DE)

(73) Assignee: ATRIVA THERAPEUTICS GMBH, Tuebingen (DE)

(21) Appl. No.: 17/920,704

(22) PCT Filed: Apr. 22, 2021
(86) PCT No.: PCT/EP2021/060490

§ 371 Date: Oct. 21, 2022

(30) Foreign Application Priority Data
Apr. 22, 2020 (LU) LU101746

(51) Int. Cl. A61K 31/551; A61P 31/16; A61P 11/00; G01N 33/50

(52) U.S. Cl. CPC A61K 31/551; A61P 31/16; A61P 11/00; G01N 33/5044; G01N 33/5035

(57) ABSTRACT

The present invention relates to a ROCK inhibitor for use in the treatment or prevention of pulmonary edema associated with a virus infection. The present invention further concerns a use of an in vitro test system or the determination of inhibitors effectiveness in preventing or reducing apical sodium-potassium-ATPase (NKA) localisation in lung epithelial cells. Also provided is a method for detecting molecules effective in the prophylaxis and/or treatment of a pulmonary edema. Finally, the invention relates to a test system.

### TECHNICAL FIELD OF THE INVENTION

The present invention relates to a ROCK inhibitor for use in the treatment or prevention of pulmonary edema associated with a virus infection. The present invention further concerns a use of an in vitro test system or the determination of inhibitor's effectiveness in preventing or reducing apical sodium-potassium-ATPase (NKA) localisation in lung epithelial cells. Also provided is a method for detecting molecules effective in the prophylaxis and/or treatment of a pulmonary edema. Finally, the invention relates to a test system.

### DESCRIPTION

Pulmonary edema can be caused by many different factors. It can be related to heart failure, called cardiogenic pulmonary edema, or related to other causes such as viral infections, referred to as non-cardiogenic pulmonary edema. For example, the Influenza A Virus (IAV) infection of humans can lead to lung damage and the “Acute Respiratory Distress Syndrome” (ARDS), which is caused by the excessive accumulation of fluid (pulmonary edema) in the alveolar lung space and can lead to hypoxemia and death without treatment.

Effective treatment requires prompt diagnosis and early intervention. Consequently, over the past 2 centuries a concentrated effort to develop clinical tools to rapidly diagnose pulmonary edema and track response to treatment has occurred. The ideal properties of such a tool would include high sensitivity and specificity, easy availability, and the ability to diagnose early accumulation of lung water before the development of the full clinical presentation. In addition, clinicians highly value the ability to precisely quantify extravascular lung water accumulation and differentiate hydrostatic from high permeability etiologies of pulmonary edema.

Therefore, there still exists a need in the art for therapies of pulmonary edema.

The solution of the present invention is described in the following, exemplified in the examples, illustrated in the figures and reflected in the claims.

The present invention relates to a ROCK inhibitor for use in the treatment or prevention of pulmonary edema by
i) preventing apical NKA localisation in lung epithelial cells, or
ii) reducing apical NKA localisation in lung epithelial cells compared to the apical NKA localisation before the administration of the ROCK inhibitor,
   wherein the pulmonary edema is associated with a virus infection, and wherein the virus is of the order Articulavirales (e.g. Orthomyxoviridae), Mononegavirales (e.g. Pneumoviridae) and/or Bunyavirales (e.g. Hantaviridae).

The present invention also relates to a composition comprising a ROCK inhibitor for use in a method for the prophylaxis and/or treatment of pulmonary edema by
i) preventing apical NKA localisation in lung epithelial cells, or
ii) reducing apical NKA localisation in lung epithelial cells compared to the apical NKA localisation present before the administration of the ROCK inhibitor,
   wherein the pulmonary edema is associated with a virus infection, and wherein the virus is of the order Articulavirales, Mononegavirales and/or Bunyavirales.

The invention also relates to the use of an in vitro test system comprising cultured lung epithelial cells infected with a virus of the order Articulavirales, Mononegavirales and/or Bunyavirales for the determination of inhibitors effective in preventing or reducing virus-induced apical NKA localisation in lung epithelial cells.

Further, the present invention concerns a method for detecting molecules effective in the prophylaxis and/or treatment of a pulmonary edema comprising contacting an in vitro test system comprising cultured lung epithelial cells infected with a virus of the order Articulavirales, Mononegavirales and/or Bunyavirales with a compound of interest, wherein the compound of interest reduces apical NKA localisation in lung epithelial cells, compared to the in vitro test system before the contacting.

The present invention also relates to a method of treating a subject having or being at risk of pulmonary edema by preventing RNA virus associated apical NKA localisation in lung epithelial cells.

The present invention also relates to a test system comprising i) a ROCK inhibitor; ii) lung epithelial cells iii) a virus of the order Articulavirales, Mononegavirales and/or Bunyavirales; and iv) means for the detection of NKA.

The Figures show:

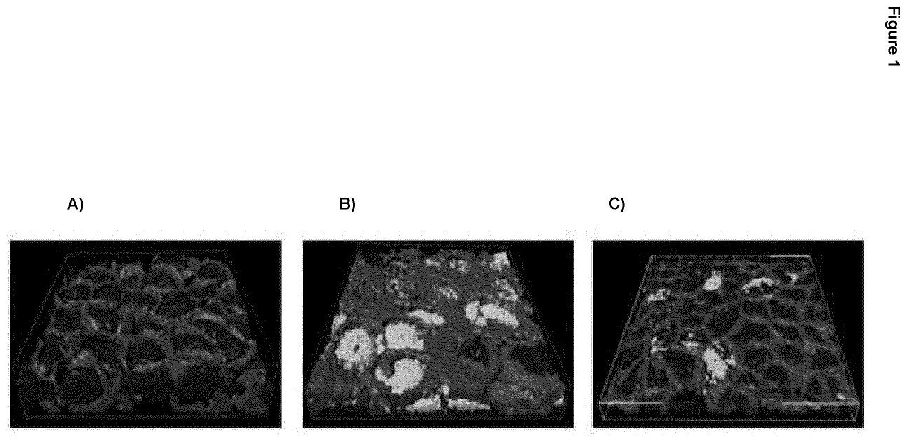

FIG. 1: ROCK inhibition prevents apical sodium-potassium-ATPase (NKA) localization. Control: Basolateral distribution of NKA (light grey) in non-infected Calu3 cells. PR8: Apical NKA distribution in Calu3 cells infected with influenza virus A/Puerto Rico/8/34 (H1N1), 20 h p.i. PR8+Rho XIII: NKA distribution in Calu3 cells infected with influenza virus A/Puerto Rico/8/34 and treated with ROCK inhibitor (20 h post infection (p.i.)). (NKAα-(dark grey), viral nucleoprotein (NP) (light grey), nucleus (dark grey). Section thickness: 0.25 μm, scale bar: 10 μm.

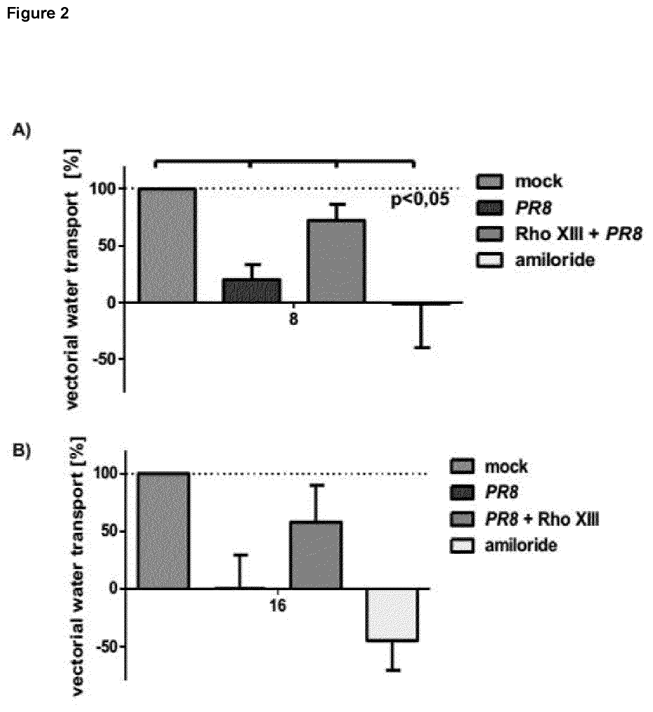

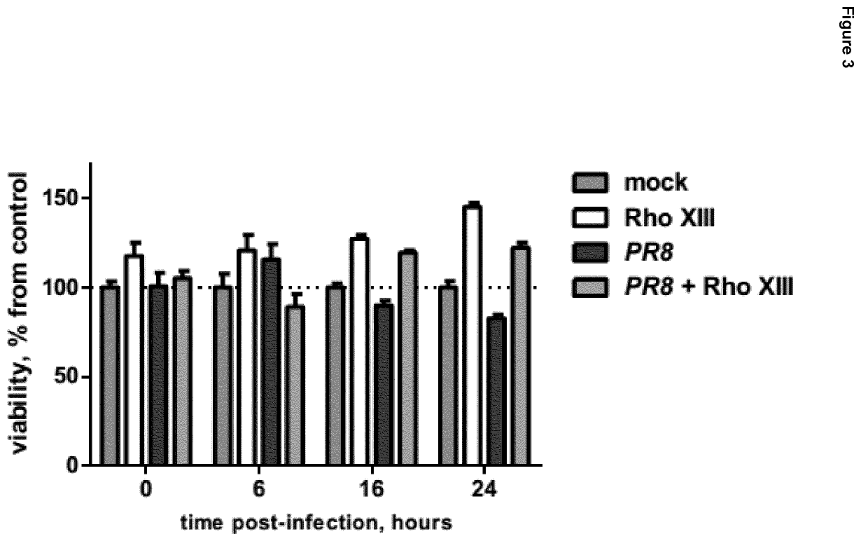

FIG. 2: ROCK inhibition improves vectorial water transport in polarized (infected) Calu3 cells: Mock: Vectorial water transport (VWT) over untreated and uninfected polarized Calu3 cells was measured and set to 100%. PR8: The VWT via untreated and influenza virus A/Puerto Rico/8/34 (H1N1, MOI=2)-infected Calu3 cells. Rho XIII+PR8: The VWT via treated (ROCK inhibitor: XIII, 5 μM) and influenza virus A/Puerto Rico/8/34 (H1N1)-infected Calu3 cells. Amilorides: The VWT via amiloride (blocks ENaC)-treated and non-infected Calu3 cells (negative control, shows the ENaC-dependent portion). A) 8 hours p.i., B) 16 hours p.i.

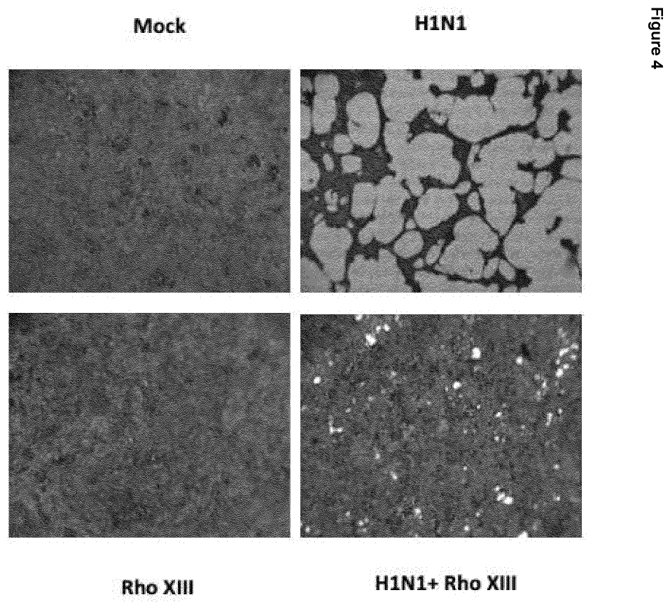

FIG. 3: ROCK inhibition increases cell viability of non-infected and infected Calu3 cells. Mock: Calu3 cells were incubated for 0, 6, 16, 24 hours and the viability was determined (MTT test) and set to 100%. Rho XIII: Calu3 cells were treated for 0, 6, 16, 24 hours with the ROCK inhibitor Rho XIII (5 μM) and subsequently viability was determined. H1N1: Calu3 cells were infected with influenza virus A/Puerto Rico/8/34 (H1N1) (MOI=2) and viability was determined. Rho XIII+H1N1: Calu3 cells infected with influenza virus A/Puerto Rico/8/34 (H1N1) and treated for 0, 6, 16, 24 hours with the ROCK inhibitor Rho XIII (5 μM) and subsequently viability was determined.

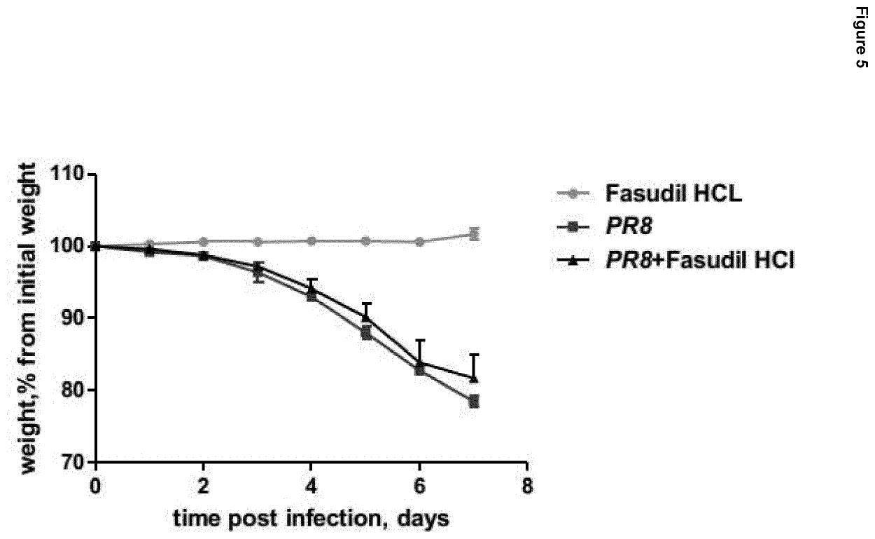

FIG. 4: ROCK inhibition reduces virus-induced epithelial cell damage in vitro. Mock: Confluently grown, polarized, mock-infected Calu3 cells were stained with Coomassie (blue). H1N1: Cells were infected with influenza virus A/Puerto Rico/8/34 (MOI=5) and stained 24 h p.i. with Coomassie. Rho XIII: confluently grown Calu3 cells were treated with ROCK inhibitor (Rho XIII, 5 μM) and stained with Coomassie 24 hours later. H1N1+Rho XIII: Confluent grown Calu3 cells were infected with influenza virus A/Puerto Rico/8/34 and treated with ROCK inhibitor (Rho XIII, 5 μM) and stained with Coomassie 24 h p.i.

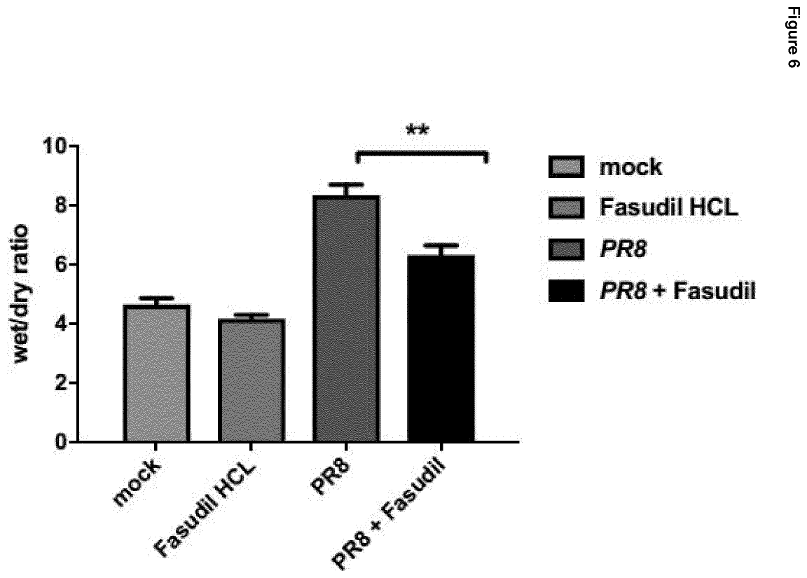

FIG. 5: ROCK inhibition leads to a tendential reduction of the body weight loss of infected C57BL/6 mice. Fasudil HCL: body weight from non-infected mice which had been treated 1×daily with Fasudil HCl (10 mg/kg in saline solution). PR8: Body weight of mice infected with IAV A/Puerto Rico/8/32 (H1N1, 500 PFU/mouse). PR8+Fasudil HCL: body weight of mice infected with IAV A/Puerto Rico/8/32 (H1N1, 500 PFU/mouse) and treated 1×daily with Fasudil HCl (10 mg/kg in saline solution). The experiment was planned for 7 days only. Afterwards the mice were euthanized for ethical reasons. Therefore, it is possible that the black and the red curve would separate even more strongly (significantly) from each other.

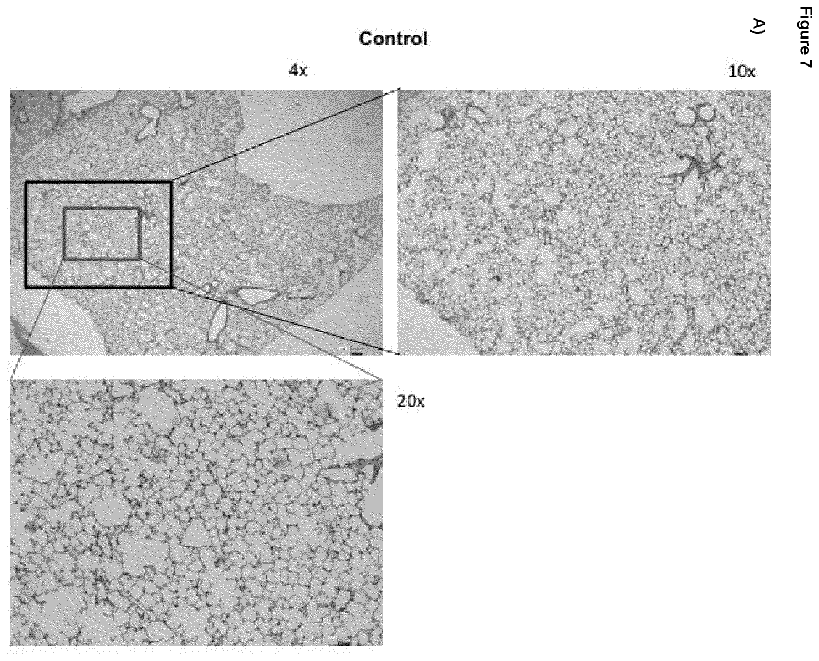

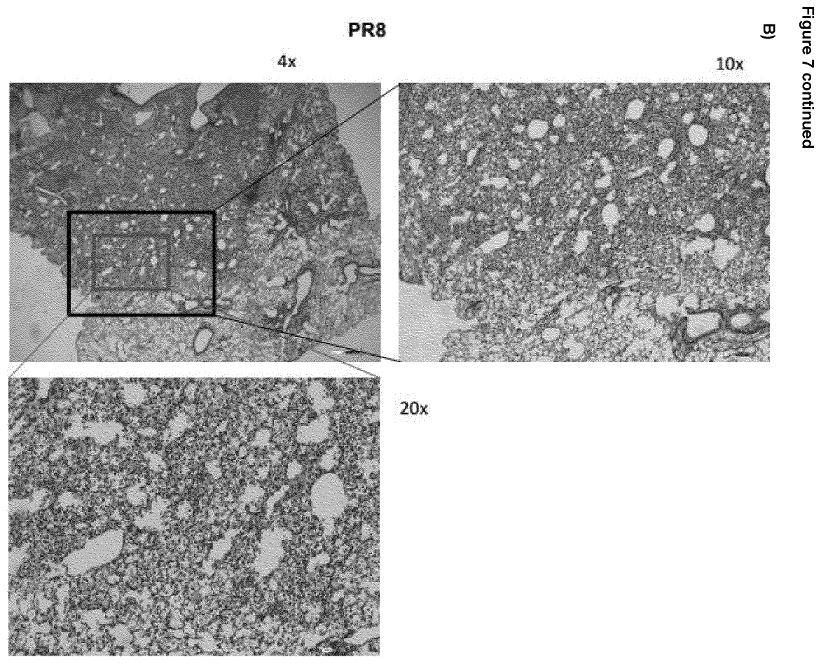

FIG. 6: ROCK inhibition reduces the fluid weight (wet dry weight ratio) of the infected mouse lung. Mock: Wet-dry lung ratio of untreated, mock-infected mice. Fasudil HCL: Wet-dry ratio of lung of mice treated 1×daily for 6 days with Fasudil HCl (10 mg/kg in saline solution). PR8: Wet-dry lung ratio of mice infected with IAV A/Puerto Rico/8/32 (H1N1, 500 PFU/mouse) (7th day p.i.). PR8+Fasudil HCL: Wet-dry lung ratio of mice infected with IAV A/Puerto Rico/8/32 (H1N1, 500 PFU/mouse) and treated 1×daily for 6 days with Fasudil HCl (10 mg/kg in saline solution) (7th day p.i.).

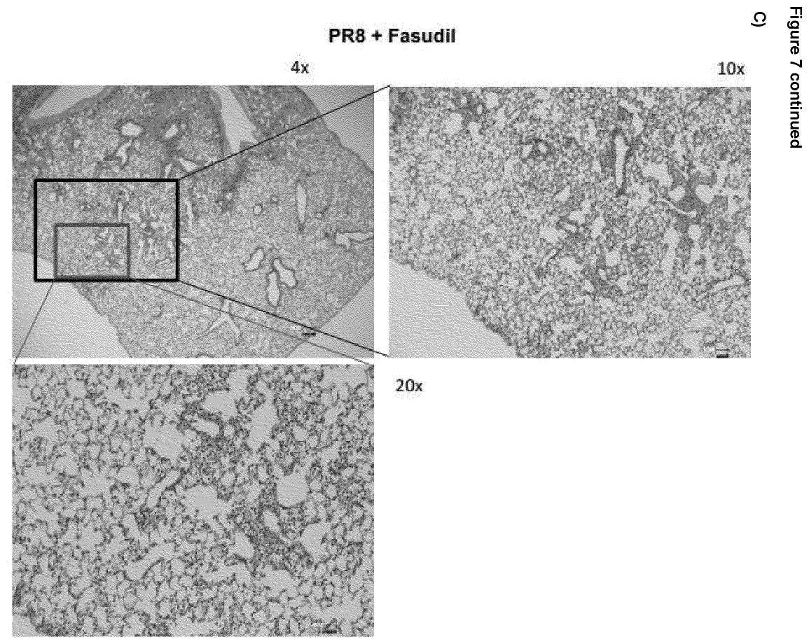

FIG. 7: ROCK inhibition improves the tissue structure of the lung and reduces the cellular alveolar infiltration of infected mice. Control: Lung tissue section (various enlargements) representative for animals treated 1×daily for 6 days with Fasudil HCl (10 mg/kg in saline solution) (7th day p.i.). PR8: Lung tissue section (various enlargements) representative of animals infected with IAV A/Puerto Rico/8/32 (H1N1, 500 PFU/mouse) (7th day p.i.). PR8+Fasudil HCL: Lung tissue section (various magnifications) representative of animals infected with IAV A/Puerto Rico/8/32 (H1N1, 500 PFU/Mouse) and treated once daily for 6 days with Fasudil HCl (10 mg/kg in saline solution) (7th day p.i.), showing one overview image (4×) and two magnifications of selected sections (10×, 20×).

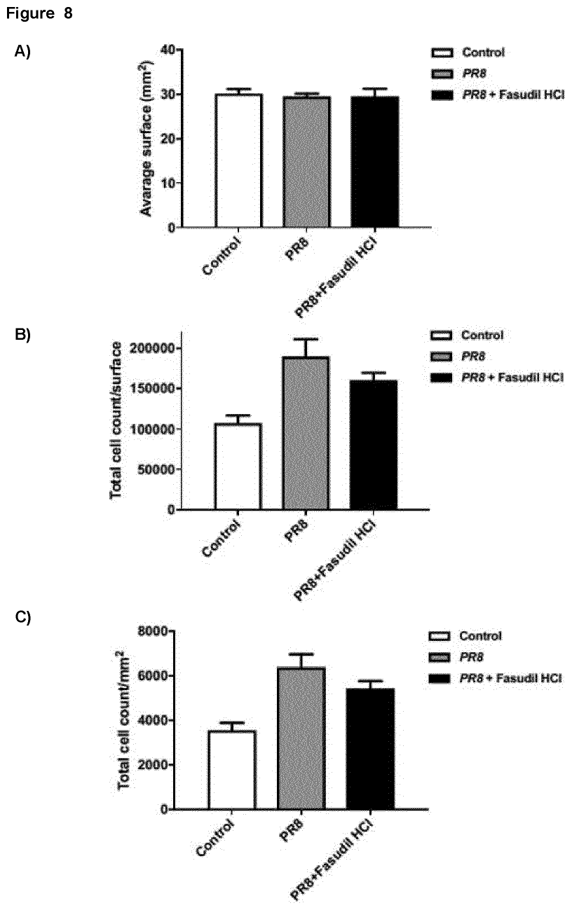

FIG. 8: ROCK inhibition reduces the number of infiltrates (quantification). Control: Lung tissue slices from 5 mice, which were treated 1×daily for 6 days with Fasudil HCl (10 mg/kg in saline solution) (7th day p.i.). PR8: Lung tissue sections from 5 mice infected with IAV A/Puerto Rico/8/32 (H1N1, 500 PFU/mouse) (7th day p.i.). PR8+Fasudil HCL: Lung tissue sections from 5 mice infected with IAV A/Puerto Rico/8/32 (H1N1, 500 PFU/mouse) and treated 1×daily for 6 days with Fasudil HCl (10 mg/kg in saline solution) (7th day p.i.). The lung tissue sections were digitized with an Aperio CS2 scanner (Leica Biosystems Imaging Inc., CA, USA) and analyzed using “Aperio v9 nuclear count algorithm” software (Leica Biosystems Imaging Inc., CA, USA) and the average area of the lung sections of each group determined (Average surface/mm2), the average total cell count/surface and the average cell count per mm 2 calculated (Total cell count/mm2).

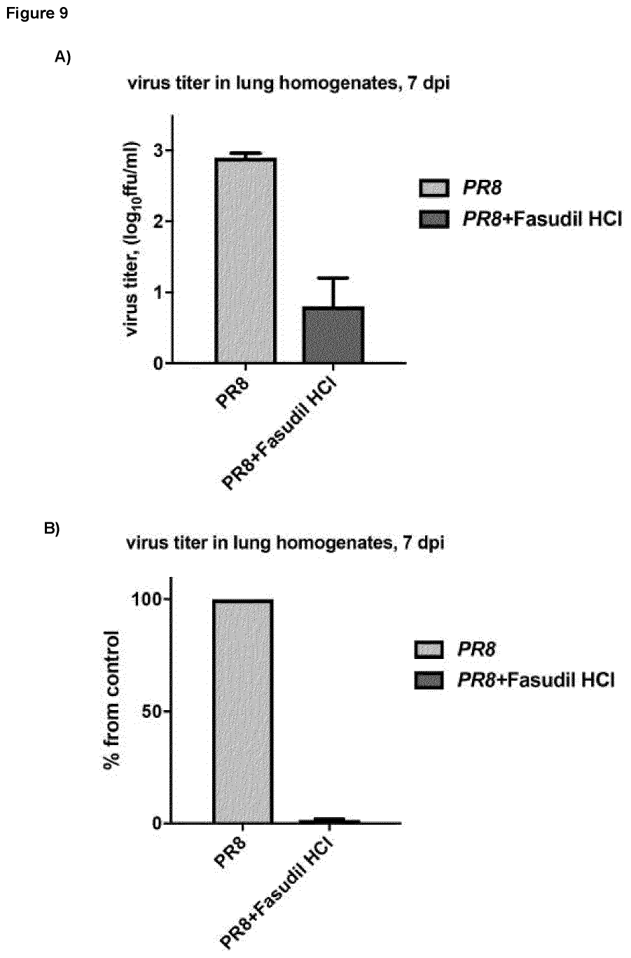

FIG. 9: ROCK inhibition reduces the virus titer in the lungs of infected mice. PR8: Virus titer of lung homogenizates from mice infected with IAV A/Puerto Rico/8/32 (H1N1, 500 PFU/Mouse) were infected (7th day p.i.). PR8+Fasudil HCL: Virus titre of lung homogenizates from mice infected with IAV A/Puerto Rico/8/32 (H1N1, 500 PFU/mouse) and treated 1×daily for 6 days with Fasudil HCl (10 mg/kg in saline solution) (7th day p.i.). n=3

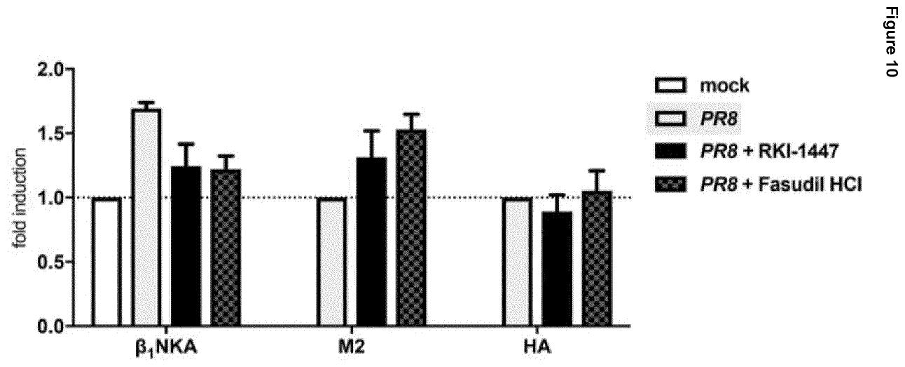

FIG. 10: Cell surface expression of NKA, M2 and HA in PR8-infected cells. Amount of NKA, M2 and HA on the cell surface of PR8-infected Calu3 cells (+/−) ROCK inhibitor treatment was assessed by On-Cell Western Blot analysis of non-permeablized cells using antibodies specific for NKA (beta-subunit, HA and M2 (n=16).

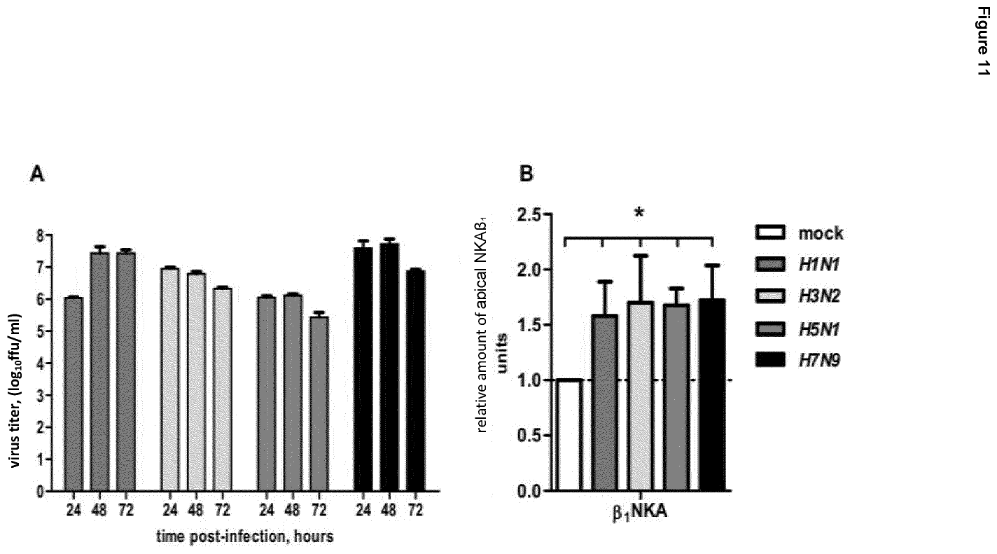

FIG. 11: Redistribution of NKAp1 within the plasma membrane of Calu3 cells infected with different IAV subtypes.

(A) Growth of different IAV subtypes in Calu3 cells. Cells were infected with the indicated viruses at MOI 0.01 and virus titers were determined by foci assay at the indicated time points. Bar graph represents mean+SD, n=3.

(B) Quantification of NKAβ1 on the apical membrane of Calu3 cells infected with different strains of IAV. Monolayer of Calu3 cells on 96-well plate were infected with the indicated viruses (MOI:2) and 20 h p.i. OCWB analysis was performed. Data represent the mean+SD, n=16.

In healthy/control lung epithelial cells the NKA is localized essentially basolaterally. In this situation, sodium is taken up into the lung epithelial cells via e.g. apically localized sodium channels (ENaC). The NKA then exports the sodium from the lung epithelial cell into the subepithelial interstitial space. Water follows this sodium gradient into the subepithelial interstitial space via aquaporins and via other intra-cellular routes. In this way, the development of pulmonary edema is prevented. It follows that the NKA represents a major limiting factor in edema clearance when it is functionally impaired (Peteranderl et al., (2019) “Influenza A virus infection induces apical redistribution of Na⁺, K⁺-ATPase in lung epithelial cells in vitro and in vivo” American Journal of Respiratory Cell and Molecular Biology Volume 61 Number 3, pp. 395-397)

Specifically, what the inventors have found is that upon viral infection of lung epithelial cells, preferably alveolar epithelial cells (e.g. Calu3 cells) with an influenza A virus (IAV) of the order Articulavirales and thus also plausibly of the order Mononegavirales and/or Bunyavirales the NKA is at least partially redistributed from the basolateral side to the apical side of the lung epithelial cells. Due to this redistribution the sodium gradient as well as the directional water transport from the apical to the basolateral lung cell side is disturbed. This leads to the development of pulmonary edema. Notably, tight junction complexes are not affected by this virus infection indicating that the cellular polarity is maintained (Peteranderl et al., (2019) “Influenza A virus infection induces apical redistribution of Na⁺, K⁺-ATPase in lung epithelial cells in vitro and in vivo” American Journal of Respiratory Cell and Molecular Biology Volume 61 Number 3, pp. 395-397).

It was surprisingly found that ROCK inhibitors (i) prevent IAV-induced pathological redistribution of NKA from the basolateral side to the apical side of virus-infected human polar lung epithelial cells (like Calu3 cells) and (ii) restores directed fluid transport (from apical to basolateral) in cell culture. Thus, ROCK inhibitors treat and/or prevent pulmonary edema by directly effecting intracellular water transport in IAV-infected lung epithelial cells. Especially, treatment or prevention is achieved by re-establishing intracellular directed water transport from the apical to the basolateral side into the institial space by restoring the sodium gradient through the prevention of IAV-induced apical NKA localization. Without being bound to theory the inventors believe that ROCK inhibitors not only prevent redistribution of NKA to the apical side but also basolaterally stabilize NKA.

Further, it was found that ROCK inhibitors (iii) greatly reduce the cytopathic effect (CPE) of IAV proliferation in cell culture. In addition, animal experiments have shown that ROCK inhibition (iv) leads to a reduction in the body weight loss of infected mice, (v) reduces the fluid weight of the infected mouse lung, (vi) stabilizes the tissue structure of the lung, (vii) reduces cellular alveolar infiltration, and (viii) reduces the virus titer in the lungs of infected mice.

Thus, the present invention relates to a ROCK inhibitor for use in the treatment or prevention of pulmonary edema by
i) preventing apical NKA localisation in lung epithelial cells, or
ii) reducing apical NKA localisation in lung epithelial cells compared to the apical NKA localisation before the administration of the ROCK inhibitor,
   wherein the pulmonary edema is associated with a virus infection, and wherein the virus is of the order, Articulavirales (e.g. Orthomyxoviridae) Mononegavirales (e.g. Pneumoviridae) and/or Bunyavirales (e.g. Hantaviridae).

The “ROCK inhibitor” as used herein can be any suitable ROCK inhibitor. In general, a ROCK inhibitor is an inhibitor of Rho-associated protein kinase (ROCK) pathway. The ROCK pathway is known to the skilled person and inter alia described by Liao et al. (2007) “Rho Kinase (ROCK) Inhibitors” J. Cardiovasc Pharmacol. 50(1):17-24 and Amano et al. (2010) “Rho-Kinase/ROCK: A Key Regulator of the Cytoskeleton and Cell Polarity” Cytoskeleton (Hoboken). 2010 September; 67(9): 545-554. Thus, the term “ROCK signaling pathway” refers to the cascade of cellular events initiated by an (active) Rho-associated kinase (Rho-kinase/ROCK/ROK).

The cascade of cellular events can e.g. start from Rho. The Rho subfamily is a member of small molecule G protein in the Ras family and has GTPase activity. Thus, Rho converts between an activated state (Rho-GTP) and an inactive state (Rho-GDP). Upon stimulation of Rho e.g. via lysophosphatic acid (LPA) or sphingosine-1 phosphate (S1P) GTP-bound Rho (active Rho) is generated. One effector molecule downstream of Rho is the Rho-associated kinase (Rho-kinase/ROCK/ROK). Thus, Rho-GTP can activate ROCKs. However, ROCKs may also be activated independently of Rho, namely through e.g. amino-terminal transphosphorylation. Upon activation the ROCK protein phosphorylates many downstream targets such as F-actin.

On the other hand, ROCKs may be inhibited by other small GTP-binding proteins, such as Gem and Rad.

Specifically, ROCKs consist of an amino-terminal protein serine/threonine kinase domain, followed by a mid coiled-coil-forming region containing a Rho-binding domain (RBD), and a carboxy-terminal cysteine-rich domain (CRD) located within the pleckstrin homology (PH) motif as also described by Liao et al. (2007) “Rho Kinase (ROCK) Inhibitors” J. cardiovasc Pharmacol. 50(1):17-24. So far two ROCK isoforms have been identified, namely ROCK1 and ROCK2.

An “inhibitor” of ROCK as used herein, is defined as any suitable inhibitor capable of a decreasing or inhibiting the activity of a ROCKs or the ROCK pathway. The inhibitor may be a compound/molecule decreasing or abolishing the activity of a ROCKs or the ROCK pathway. The inhibitor may achieve this effect by decreasing or blocking the transcription of the gene encoding the ROCK and/or decreasing the translation of the mRNA encoding the ROCK. It can also be that the inhibitor leads to that ROCK(s) performs its biochemical function with decreased efficiency in the presence of the inhibitor than in the absence of the inhibitor. Further, it is possible that the inhibitor results in that ROCK performs its cellular function with decreased efficiency in the presence of the activator than in the absence of the inhibitor.

Accordingly, the term “inhibitor” also encompasses molecules/compounds that have a directly decreasing effect on the ROCK pathway e.g. Gem and Rad but also molecules that are indirectly decreasing, e.g. by interacting for example with molecules that positively regulate (e.g. activate such as Rho-GTP, LPA or S1P) the ROCK pathway.

The inhibitor can also be an antagonist of the pathway to be inhibited.

Methods for testing if a compound/molecule is capable to decrease or inhibit the activity of a ROCK or the ROCK pathway are known to the skilled person. For example, an inhibitor of the ROCK pathway or ROCK can be tested by performing standard tests, known to the skilled person. The skilled person may contact a probe with myosin phosphate target subunit 1 (MYPT1). MYPT1 is a substrate of ROCK1 and ROCK2 that phosporylates this substrate at threonine 696 (T696). This posphorylation may be detected by an anti-phopsho-MYPT1-Thr696 antibody. A recombinant active ROCK2 may be used as a positive control. Such tests are commercially available and can e.g. be obtained from abcam (ROCK Activity Assay; abcam Cat #ab211175) or Millipore (Millipore Cat #CSA001).

An inhibitor may inhibit or decrease the ROCK pathway or ROCK activity by 10%, 20%, 30%, 40%, 50%, 60%, 70%, 80%, 90%, 100% or more when compared to the activity of the ROCK pathway or ROCK activity without or before the addition of the inhibitor.

The ROCK inhibitor may be a small molecule, a compound, a binding molecule such as an antibody, a nucleic acid molecule such as a siRNA or a prodrug.

As used herein the “small molecule” can be any small molecule that can decrease/inhibit the ROCK pathway (Rho/ROCK pathway) or ROCK activity. The small molecule can be an organic compound of low molecular weight. Low molecular weight may mean that the compound has a weight of less than 900 daltons (da), less than 800 da, less than 700 da, less than 600 da or less than 500 da. For example, the small molecule may have a molecular weight of about 300 da. The size of a small molecule can be determined by methods well-known in the art, e.g., mass spectrometry. Exemplary small molecule ROCK inhibitors include Y-27632 and CCG-1423 as well as Fasudil HCl (327.830898 Da), RKI-1447 (326.374812 Da) and Hydroxyfasudil (307.369824 Da).

As used herein the “compound” can be any compound that can decrease/inhibit the ROCK pathway (Rho/ROCK pathway) or ROCK. The compound/molecule that can be used as an inhibitor can be any compound/molecule, which can inhibit or decrease the respective pathway/ROCK or which activates a suppressor of the pathway/ROCK or inhibits an activator of the pathway/ROCK to be inhibited.

As used herein the “binding molecule” can be any binding molecule that can decrease/inhibit the ROCK pathway (Rho/ROCK pathway) or ROCK. For example, the binding molecule can be an antibody, an antibody fragment or a divalent antibody fragment comprising two binding sites with different specificities.

The antibody can be any anti-ROCK1 and/or anti-ROCK2 antibody. Such antibodies are commercially available. For example, the antibody can be the anti-ROCK1 antibody [EP786Y] or [EPR638Y] of abcam (ab45171 or ab230799), the anti-ROCK2 antibody of abcam (ab71598), the Rock1 antibody (B-1) of Santa Cruz (sc-374388), the anti-anti-ROCK1 antibody (C8F7) mAb #4035 from Cell Signaling, the anti-ROCK2 antibody (D1B1) mAb #9029 from Cell Signaling or the anti-ROCK2 Antibody #8236 from Cell Signaling.

Non limiting examples of such divalent antibody fragments include a (Fab)₂′-fragment, a divalent single-chain Fv fragment, a bsFc-1/2-dimer or a bsFc-CH3-1/2 dimer. The binding molecule may also only have a single binding site, i.e., may be monovalent. Examples of monovalent binding molecules include, but are not limited to, a monovalent antibody fragment, a proteinaceous binding molecule with antibody-like binding properties. Examples of monovalent antibody fragments include, but are not limited to a Fab fragment, a Fv fragment, a single-chain Fv fragment (scFv) or an scFv-Fc fragment.

Alternatively, the binding molecule can also be a bivalent proteinaceous artificial binding molecule such as a lipocalin mutein that is also known as “duocalin”.

The binding molecule can also be a proteinaceous binding molecule with antibody-like binding properties. Exemplary but non-limiting proteinaceous binding molecules include an aptamer, a mutein based on a polypeptide of the lipocalin family, a glubody, a protein based on the ankyrin scaffold, a protein based on the crystalline scaffold, an adnectin, an avimer or a (recombinant) receptor protein.

The inhibitor can also be a nucleic acid molecule. Examples include RNA, siRNA, miRNA or a non-proteinaceous aptamer that can inhibit the ROCK pathway (Rho/ROCK pathway) or ROCK. Such an aptamer is an oligonucleic acid that binds to a specific target molecule. These aptamers can be classified as: DNA or RNA aptamers. They consist of (usually short) strands of oligonucleotides. Also, the nucleic acid molecules may be used to suppress an activator, promoter or enhancer of a pathway to be inhibited.

siRNAs for ROCK1 and ROCK2 are commercially available to the skilled person. For example, the siRNA can be the Rock-1 siRNA (h) of Santa Cruz (sc-76025), ROCK2 siRNA (h) of Santa Cruz (sc-29474), ROCK-1 siRNA (h) #4390824 of Thermos Fischer Scientific or ROCK-2 siRNA (h) AM51331 of Thermo Fischer Scientific.

The inhibitor can also be a prodrug that can inhibit the ROCK pathway (Rho/ROCK pathway)/ROCK. A “prodrug” is pharmacologically essentially inactive and is metabolized in the body of the subject that has been administered with the prodrug into its active form. Suitable prodrugs are for example described in WO2012/015760.

It is also envisioned that the ROCK inhibitor is a ROCK1 and/or ROCK2 inhibitor. An exemplary ROCK1 inhibitor is Fasudil as described herein. An exemplary ROCK1 and ROCK2 inhibitor is RhoXIII. An exemplary ROCK2 inhibitor is SLx-2119 (also known as KD025). The ROCK inhibitor may be any inhibitor as listed in Table 1 or combinations thereof.

Exemplary inhibitors of the ROCK therefore include, but are not limited to, Fasudil, Y27632 (CAS: 146986-50-7), Hydroxyfasudil, H-1152-P (Dimethylfasudil), Y27632, Y30141, Y32885 (Wf536), Y39983 (CAS: 203911-26-6), DW1865, SLx-2119 (CAS: 911417-87-3), SR8046, SR6246, Ripasudil, AS1892892, AR12141, AR12432, INS-117548, INS-115644, AT13148, RK11447, SAR407899, Netarsudil (a.k.a. AR-13324), AR12286, PG286*, PG324**, ATS907, AMA0076, Thiazovivin, Azabenzimidazole-aminofurazans, H-0104 Dihydrochloride (CAS: 913636-88-1), DE-104, Olefins, Isoquinolines (CAS: 119-65-3), Indazoles (CAS: 271-44-3), pyridinealkene derivatives, H-1152 dichloride (CAS: 871543-07-6), XD-4000, HMN-1152, 4-(1-aminoalkyl)-N-(4-pyridyl)cyclohexane-carboxamides (Oral formulation), Rhostatin, BA-210, BA-207, BA-215, BA-285, BA-1037, Ki-23095, VAS-012, quinazoline, AR13154, AMA0428 and/or Rho XIII or combinations thereof.

The ROCK inhibitor can also be selected from Fasudil, Hydroxyfasudil, Dimethylfasudil, Y27632, Y30141, Y32885, Y39983, DW1865, SLx-2119, SR8046, SR6246, Ripasudil, AS1892892, AR12141, AR12432, INS-117548, INS-115644, AT13148, RK11447, SAR407899, Netarsudil, AR12286, ATS907, AMA0076, Thiazovivin, H-0104 Dihydrochloride, Olefins, Isoquinolines, Indazoles, H-11152 dichloride, 4-(1-aminoalkyl)-N-(4-pyridyl) cyclohexane-carboxamides, quinazoline, Rho XIII, AR13154, AMA0428 and/or or combinations thereof.

The ROCK inhibitor can also be selected from Fasudil, Y27632, Y39983, SLx-2119, Ripasudil, INS-117548, INS-115644, AT13148, SAR407899, Netarsudil, AR12286, ATS907, Rho XIII and/or or combinations thereof.

The ROCK inhibitors described herein are further characterized in below Table.

TABLE 1
List of ROCK inhibitors and further information such as structural formel,
clinical trial numbers and CAS numbers.

| # | Name | Srtucture or other identification |
|---|---|---|
| 1 | Fasudil | 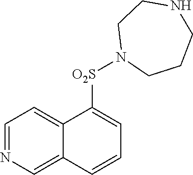 [SMILES: O=S(=O)(c1cccc2cnccc12)N1CCCNCC1] |
| 2 | Hydroxyfasudil | 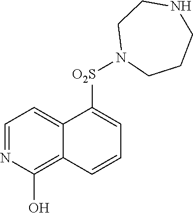 [SMILES: O=S(=O)(c1cccc2c(O)nccc12)N1CCCNCC1] |
| 3 | H-1152-P (Dimethylfasudil) | 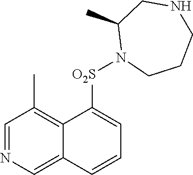 [SMILES: Cc1cncc2cccc(S(=O)(=O)N3CCCNC[C@@H]3C)c12] |
| 4 | Y27632 | 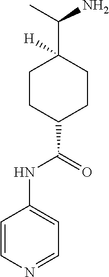 |
| 5 | Y30141 | 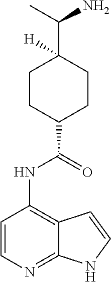 |
| 6 | Y32885 (Wf536) | 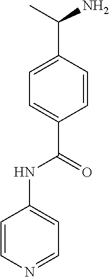 [SMILES: C[C@@H](N)c1ccc(C(=O)Nc2ccncc2)cc1] |
| 7 | Y39983 (also named RKI-983, SNJ-1656) | 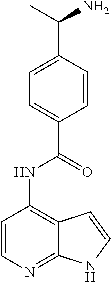 [SMILES: C[C@@H](N)c1ccc(C(=O)Nc2ccnc3[nH]ccc23)cc1] |
| 8 | DW1865 | 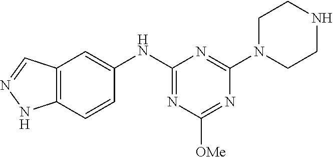 [SMILES: COc1nc(Nc2ccc3[nH]ncc3c2)nc(N2CCNCC2)n1] |
| 9 | SLx-2119 (KD025) | 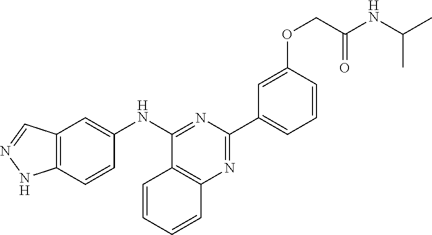 [SMILES: CC(C)NC(=O)COc1cccc(-c2nc(Nc3ccc4[nH]ncc4c3)c3ccccc3n2)c1] |
| 10 | SR8046 | 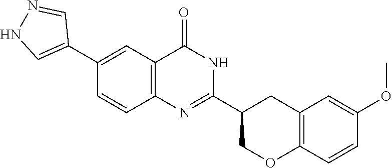 [SMILES: COc1ccc2c(c1)C[C@H](c1nc3ccc(-c4cn[nH]c4)cc3c(=O)[nH]1)CO2] |
| 11 | SR6246 | 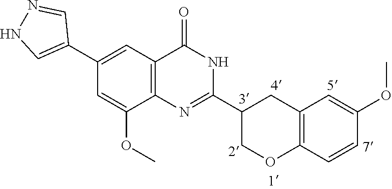 [SMILES: COc1ccc2c(c1)CC(c1nc3c(OC)cc(-c4cn[nH]c4)cc3c(=O)[nH]1)CO2] |
| 12 | Ripasudil | 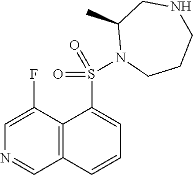 [SMILES: C[C@H]1CNCCCN1S(=O)(=O)c1cccc2cncc(F)c12] |
| 13 | AS1892892 | 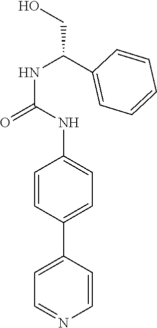 [SMILES: O=C(Nc1ccc(-c2ccncc2)cc1)N[C@H](CO)c1ccccc1] |
| 14 | AR12141 | 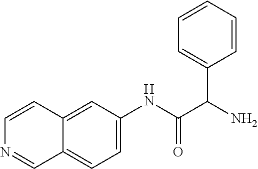 [SMILES: NC(C(=O)Nc1ccc2cnccc2c1)c1ccccc1] |
| 15 | AR12432 | 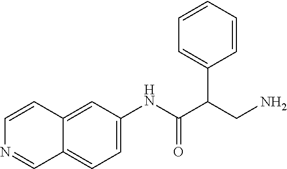 [SMILES: NCC(C(=O)Nc1ccc2cnccc2c1)c1ccccc1] |
| 16 | INS-117548 | 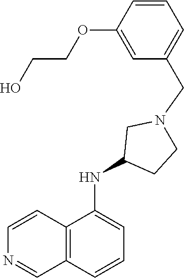 |
| 17 | INS-115644 (also named latrunculin B) | Generated by Merck, Whitehouse Station, NJ, USA |
| | | 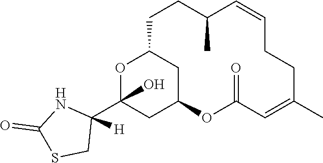 [SMILES: C/C1=C/C(=O)O[C@@H]2C[C@@H](CC[C@H](C)/C=C\CC1)O[C@@](O)([C@@H]1CSC(=O)N1)C2] |
| 18 | AT13148 | 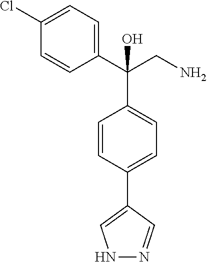 [SMILES: NC[C@@](O)(c1ccc(Cl)cc1)c1ccc(-c2cn[nH]c2)cc1] |
| 19 | RKI1447 | 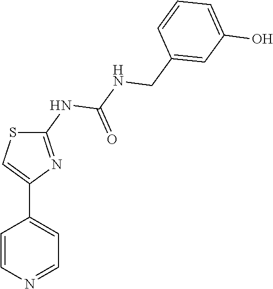 [SMILES: O=C(NCc1cccc(O)c1)Nc1nc(-c2ccncc2)cs1] |
| 20 | SAR407899 | 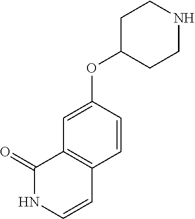 [SMILES: O=c1[nH]ccc2ccc(OC3CCNCC3)cc12] |
| 21 | Netarsudil (a.k.a. AR-13324) | 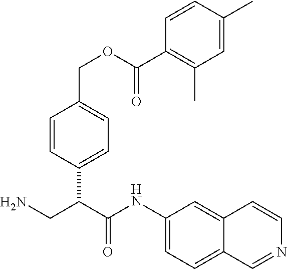 [SMILES: Cc1ccc(C(=O)OCc2ccc([C@@H](CN)C(=O)Nc3ccc4cnccc4c3)cc2)c(C)c1] |
| 22 | AR12286 | 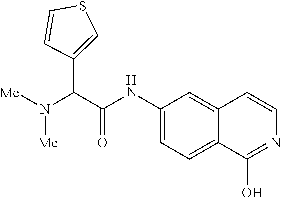 [SMILES: CN(C)C(C(=O)Nc1ccc2c(O)nccc2c1)c1ccsc1] |
| 23 | PG286* | ClinicalTrials.gov Identifier: NCT01789736 |
| | | Generated by Aerie Pharmaceuticals Inc. |
| 24 | PG324** | ClinicalTrials.gov identifier (NCT number): NCT02057575 |
| | | Generated by Aerie Pharmaceuticals Inc. |
| 25 | ATS907 | ClinicalTrials.gov Identifier: NCT01668524 |
| | | Generated by Altheos Inc., South San Francisco, CA, USA |
| | | 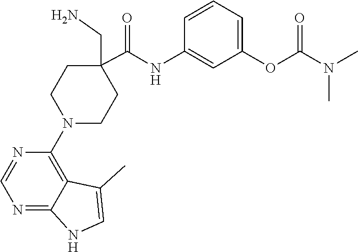 [SMILES: Cc1c[nH]c2ncnc(N3CCC(CN)(C(=O)Nc4cccc(OC(=O)N(C)C)c4)CC3)c12] |
| 26 | AMA0076 | 3-[2-(Aminomethyl)-5-[(pyridin-4-yl) |
| | | carbamoyl]phenyl] benzoate |
| | | Generated by Amakem, Belgium |
| | | ClinicalTrials.gov identifier NCT02136940 |
| 27 | DE-104 | ClinicalTrials.gov Identifier: NCT00868894 |
| | | Generated by Santen-Ube, Osaka City and Ube City, Japan |
| 28 | Thiazovivin | 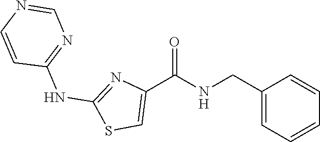 [SMILES: O=C(NCc1ccccc1)c1csc(Nc2ccncn2)n1] |
| 29 | Azabenz-imidazole- | |
| | amino--furazans | |
| 30 | H-0104 | CAS: 913636-88-1 |
| | Dihydrochloride | |
| 31 | Olefins such as ethen, cyclohexen or 1,4- pentadien | 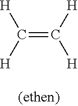 |
| | | 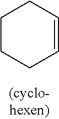 |
| | | 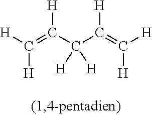 |
| 32 | Isoquinolines | CAS: 119-65-3 |
| 33 | Indazoles (CAS: 271-44-3) | 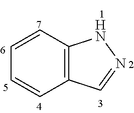 |
| 34 | Pyridine-alkene | |
| | derivatives | |
| 35 | H-1152 dichloride | CAS: 871543-07-6 |
| 36 | XD-4000 | |
| 37 | HMN-1152 | |
| 38 | 4-(1-aminoalkyl)-N- (4-pyridyl) cyclohexane- carboxamides (Oral formulation) | 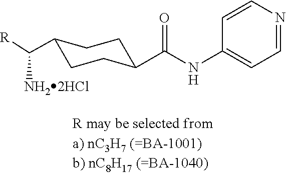 |
| | | 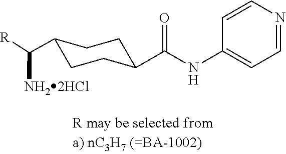 |
| 39 | Rhostatin | |
| 40 | BA-210 | |
| 41 | BA-207 | |
| 42 | BA-215 | |
| 43 | BA-285 | |
| 44 | BA-1037 | |
| 45 | Ki-23095 | |
| 46 | VAS-012 | |
| 47 | quinazoline | 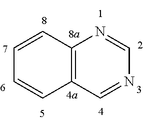 |
| 48 | Rho XIII | 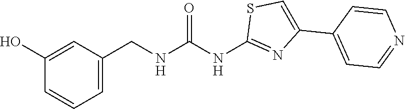 [SMILES: O=C(NCc1cccc(O)c1)Nc1nc(-c2ccncc2)cs1] |
| 49 | AR13154 | Developed by Aerie Pharmaceuticals |
| 50 | AMA0428 | Developed by Amakem |

The ROCK inhibitor can also be selected from Fasudil, Y27632, Hydroxyfasudil, Y39983, SLx-2119, Ripasudil, ATS907, INS-117548, AT13148, SAR407899, Netarsudil, AR12286, or combinations thereof.

The ROCK inhibitor can also be selected from Fasudil, Y27632, Hydroxyfasudil, Y39983, SLx-2119, Ripasudil, INS-117548, INS-115644, ATS907, AT13148, SAR407899, Netarsudil, AR12286, PG286*, PG324**, ATS907, AMA0076 and DE-104 or combinations thereof.

The ROCK inhibitor may also be a ROCK inhibitor as disclosed in WO 2009/158587 and/or in Feng et al. (2015) “Rho Kinase (ROCK) Inhibitors and Their Therapeutic Potential’ including the information about clinical trials” J. Med. Chem. 2016, 59, 6, 2269-2300.

Further, the ROCK inhibitor may be a salt of any suitable ROCK inhibitor described herein.

Thus, the ROCK inhibitor can be fausidil or a salt thereof such as fasudil HCl (Isoquinoline 5-[(hexahydro-1H-1,4-diazepin-1-yl)sulfonyl]-N-(5-Isoquinolinesulfonyl)-1,4-per-hydrodiazepine; CAS No. 103745-39-7). Fasudil HCl has the chemical formula I:

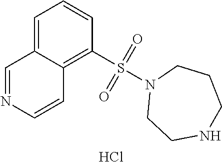 [SMILES: O=S(=O)(c1cccc2cnccc12)N1CCCNCC1 ; Cl]

Fasudil is a potent and selective inhibitor of Rho kinase, in particular ROCK2 (Ono-Saito N, et al. (1999) “H-series protein kinase inhibitors and potential clinical applications” Pharmacol Ther, 82(2-3), 123-131).

The ROCK inhibitor can also be an inhibitor of both Rho-kinase 1 and Rho-kinase 2 (ROCK1 and ROCK2), like Rho XIII (1-(3-Hydroxybenzyl)-3-(4-(pyridin-4-yl)thiazol-2-yl)urea; CAS: 1342278-01-6).

Rho XIII has the chemical formula II:

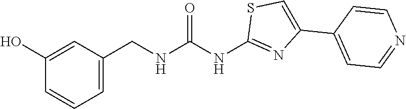 [SMILES: O=C(NCc1cccc(O)c1)Nc1nc(-c2ccncc2)cs1]

Therefore, the ROCK inhibitor may also be Fasudil and/or RhoXIII.

The ROCK inhibitor according to the present invention can be used in a method for treating or preventing pulmonary edema. As such the term “treating” or “treatment” includes administration of a ROCK inhibitor, preferably in the form of a medicament, to a subject, defined elsewhere herein, suffering from a pulmonary edema associated with a virus infection, for the purpose of ameliorating or improving symptoms.

Further, the ROCK inhibitor can act by reducing the virus (Articulavirales, Mononegavirus and/or Bunyavirales) induced apical NKA localisation in lung epithelial cells compared to the apical NKA localisation before the administration of the ROCK inhibitor.

As used herein the term “reducing” means that the amount of virally induced apical NKA localization is reduced by 10%, 20%, 30%, 40%, 50%, 60%, 70%, 80%, 90%, 95% or by 100% in the presence of the ROCK inhibitor as described herein compared to the NKA localization present before the administration of the ROCK inhibitor.

In other words, the ROCK inhibitor reduces the amount of total apical NKA, which migrated apically as result of viral infection (Articulavirales, Mononegavirus and/or Bunyavirales) of at least about 10% in presence of the ROCK inhibitor when compared to the absence or before administration of ROCK inhibitor.

As used herein, the terms “prevent”, “prevention” or “prophylaxis”, and “preventing” refer to the reduction in the risk of acquiring or developing a pulmonary edema associated with a virus infection. Also meant by “prophylaxis” is the reduction or inhibition of the recurrence of a pulmonary edema associated with a virus infection. In particular, the ROCK inhibitor can prevent pulmonary edema by stabilizing basolateral localization of NKA.

Preferably, “prevent” means that 60%, 70%, 80%, 90%, 95% or 100% of the total NKAs are localized basolaterally. The localization of the NKA can inter alia be determined by immunostaining for the NKA as e.g. described in the Examples.

The ROCK inhibitor as defined herein is used to treat or prevent pulmonary edema. The term “pulmonary edema” is known to the skilled person and inter alia described by Matthay et al. (2019) “Acute Respiratiory distress Syndrome” Nat Rev Dis Primers, 5(1): 18, p. 1-52 as well as Mutlu et al. (2005) “mechanisms of pulmonary edema clearance” Am J Physiol Lung Cell Mol Physiol. 289: L685-695 and Assaad et al. (2018) “Assessment of Pulmonary Edema: Principles and Practice.” Journal of Cardiothoracic and Vascular Anesthesia, 32(2), 901-914. As such “pulmonary edema” refers to fluid accumulation in the tissue and air spaces of the lungs, in particular in the alveoli, the microscopic air sacs of the lungs.

Thus, pulmonary edema as used herein is characterized by the accumulation of extravascular lung water (EVLW). One factor that may cause pulmonary edema is the increase in pulmonary capillary permeability. Fluid accumulation in the lung, namely pulmonary edema, can be due to damage to the lung, which is associated with viral infection. The resulting fluid accumulation in the lungs impairs gas exchange and may lead to respiratory distress or even the need for mechanical ventilation (Assaad et al. (2018) “Assessment of Pulmonary Edema: Principles and Practice.” Journal of Cardiothoracic and Vascular Anesthesia, 32(2), 901-914).

For example, often influenza virus infection is associated with pneumonia and the so-called “acute respiratory distress syndrome” (ARDS). This is inter alia described by Ňamendys-Silva et al. (2011) “Acute respiratory distress syndrome caused by influenza B virus infection in a patient with diffuse large B-cell lymphoma” Case Rep Med. 2011; 2011: 647528. ARDS is a form of fluid accumulation in the lungs, which is typically provoked by a diffuse injury to the respiratory epithelium as also described by Matthay et al. (2019) “Acute Respiratory Distress Syndrome” Nat rev Dis primers, 5(1):18.

Pulmonary edema may be diagnosed by chest X ray or computer tomography. Chest X-ray is known to the skilled artisan. It is a projection radiograph of the chest used to diagnose conditions affecting the chest. Features useful for broadly assessing pulmonary edema on a plain chest radiograph include: central pulmonary venous congestion, upper lobe pulmonary venous diversion/pulmonary venous engorgement/stag's antler sign, increased cardiothoracic ratio/cardiac silhouette size: useful for assessing for an underlying cardiogenic cause or association. Features of pulmonary interstitial edema can include: peribronchial cuffing and perihilar haze, septal lines/Kerley lines, thickening of interlobar fissures. Features of pulmonary alveolar edema: air space opacification classically in a batwing distribution may have air bronchograms pleural effusions and fluid in interlobar fissures (including ‘vanishing’ pulmonary pseudo tumor. There is a general progression of signs on a plain radiograph that occurs as the pulmonary capillary wedge pressure (PCWP) increases. Whether all or only some of these features can be appreciated on the plain chest radiograph, depends on the specific etiology. Furthermore, pulmonary edema is usually a bilateral process, but it may uncommonly appear to be unilateral in certain situations and pathologies.

The pulmonary capillary wedge pressure (PCWP) is the pressure measured by wedging a pulmonary catheter with an inflated balloon into a small pulmonary arterial branch. It estimates the left atrial pressure. How to measure the PCWP is inter alia described by McIntyre et al. (1992) “A noninvasive method of predicting pulmonary-capillary wedge pressure” N Engl J Med. 327(24): 1715-20.

It is further envisioned that the pulmonary edema can be of grade 1. In grade 1 pulmonary vascular congestion can be detected on a chest radiograph, specifically the pedical width may be detected to be more than 53 cm and/or the PCWP can be of 12-17 mmHg (Assaad et al. (2018) “Assessment of Pulmonary Edema: Principles and Practice.” Journal of Cardiothoracic and Vascular Anesthesia, 32(2), 901-914).

It is further contemplated that the pulmonary edema can be of grade 2. In grade 2 interstitial edema can be detected on a chest radiograph, specifically Kerley B lines and/or peribronchial cuffing (thickended end on bronchioal walls) may be detected and/or the PCWP can be of 17-25 mmHg (Assaad et al. (2018) “Assessment of Pulmonary Edema: Principles and Practice.” Journal of Cardiothoracic and Vascular Anesthesia, 32(2), 901-914).

It is further envisioned that the pulmonary edema can be of grade 3. In grade 3 evidence of alveolar edema can be detected on a chest radiograph, specifically lung consolidation may be detected to be present and/or the PCWP can be of >25 mmHg.

Thus, the pulmonary edema may be of grade 1, 2 or 3. The pulmonary edema may be of grade 1. It I also envisioned that the pulmonary edema may be of grade 2. It is also contemplated that the pulmonary edema may be of grade 3 (Assaad et al. (2018) “Assessment of Pulmonary Edema: Principles and Practice.” Journal of Cardiothoracic and Vascular Anesthesia, 32(2), 901-914).

Lung edema may also be detected by lung ultrasound and/or transpulmonary thermodilution as inter alia described by Assaad et al. (2018) “Assessment of Pulmonary Edema: Principles and Practice.” Journal of Cardiothoracic and Vascular Anesthesia, 32(2), 901-914.

The present invention can require that the ROCK inhibitor prevents/reduced apical NKA localization in lung epithelial cells upon viral infection as disclosed herein.

The NKA as used herein refers to any suitable NKA. The NKA is known to the skilled person and is inter alia described by Mutlu et al. (2005) “mechanisms of pulmonary edema clearance” Am J Physiol Cell Mol phsiol 289: L685-L695. It is a heterodimeric protein composed of an α- and a β-subunit. The α-subunit cleaves high-energy phosphate bonds and has the catalytic site for the exchange of intracellular Na⁺ for extracellular K⁺. The β-subunit is a smaller glycosylated transmembrane protein that appears to control the heterodimer assembly and insertion into the plasma membrane. Both subunits are required for a functional NKA. Thus, the NKA comprises a α and a β subunit.

For example, the NKA may comprise an α subunit of any one of the sequences of SEQ ID NO. 4-7 or a sequence having 70%, 75%, 80%, 85%, 90%, 95%, 97%, 98%, 99% sequence identity to a sequence of SEQ ID NO. 4-7.

Additionally or alternatively, the NKA may comprise a β subunit of any one of the sequences of SEQ ID NO. 1-3 or a sequence having 70%, 75%, 80%, 85%, 90%, 95%, 97%, 98%, 99% sequence identity to a sequence of SEQ ID NO. 1-3.

Thus, the NKA may comprise an a subunit of any one of the sequences of SEQ ID NO. 4-7 or a sequence having 70%, 75%, 80%, 85%, 90%, 95%, 97%, 98%, 99% sequence identity to a sequence of SEQ ID NO. 4-7 and a β subunit of any one of the sequences of SEQ ID NO. 1-3 or a sequence having 70%, 75%, 80%, 85%, 90%, 95%, 97%, 98%, 99% sequence identity to a sequence of SEQ ID NO. 1-3.

In accordance with the present invention, the term “identical” or “percent identity” in the context of two or more polypeptide sequences such as SEQ ID NO: 1-15 refers to two or more sequences or subsequences that are the same, or that have a specified percentage of amino acids that are the same (e.g., at least 85%, 90%, 95%, 96%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 80% to 95% or greater sequence identity are considered to be substantially identical. Such a definition also applies to the complement of a test sequence. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Also available to those having skills in this art are the BLAST and BLAST 2.6 algorithms (Altschul Nucl. Acids Res. 25 (1977), 3389-3402). The BLASTP program for amino acid sequences uses as defaults a word size (W) of 6, an expect threshold of 10, and a comparison of both strands. Furthermore, the BLOSUM62 scoring matrix (Henikoff Proc. Natl. Acad. Sci., USA, 89, (1989), 10915; Henikoff and Henikoff (1992) ‘Amino acid substitution matrices from protein blocks.’ Proc Natl Acad Sci USA. 1992 Nov. 15; 89(22):10915-9) can be used.

For example, BLAST2.6, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments.

The NKA is located on the basolateral surface of the lung epithelial cells in e.g. healthy subjects. The NKA transports ions by consuming ATP. Specifically, it pumps Na⁺ ions out of the cell in exchange for potassium influx. In this way, it maintains Na⁺ and potassium gradients across the plasma membrane.

As used herein the expression “lung epithelial cells” means any lung epithelial cell. The skilled person knows the different epithelial cells of the lung, which are inter alia described by Rackley et al. (2012) “Building and maintaining the epithelium of the lung” The Journal of Clinical investigation, vol. 122, no. 8, pp. 2724-2730 and Crystal et al. (2008) “Airway epithelial cells” Proc Am Thorac Soc, vol. 5, pp. 772-777. In general, the lung epithelial cell is polarized. This means that the lung epithelial cell has an apical and a basal side.

The lung epithelial cell may be a tracheal epithelial cell, a bronchial epithelial cell or an alveolar epithelial cell.

The person skilled in the art can determine if a cell belongs to one of these epithelial cells. For example, the skilled person could perform immunohistochemistry to detect the expression connexin 43. Connexin 43 is expressed in alveolar epithel cells (both AT1 and AT2 cells), but also on tracheal epithelial cells and bronchial epithelial cells (Johnson and Koval (2009) “Cross-Talk Between Pulmonary Injury, Oxidant Stress, and Gap Junctional Communication.” Antioxidans and Redox Signaling, vol. 11, number 2, pp. 356-367). Connexin 43 can have a sequence as shown in SEQ ID NO. 8 or be a sequence having 70%, 80%, 90%, 95%, 99% sequence identity to a sequence of SEQ ID NO. 8.

Thus, the lung epithelial cell as described herein may be a cell that expresses Connexin 43. The lung epithelial cell may be a cell that expresses Connexin 43 as shown in SEQ ID NO. 8 or be a sequence having 70%, 80%, 90%, 95%, 99% sequence identity to a sequence of SEQ ID NO. 8.

More specifically, the “tracheal epithelial cells” can extend from about 2° to 25 branches (large airways; tracheal epithelial cells). The tracheal epithelial cells line the trachea and larger bronchi.

The “bronchial epithelial cells” can extend from 2⁶ to 2²³ branches (small airways, bronchial epithelial cell). The bronchial epithelial cells line the broncheoles and smaller bronchi.

Both, the tracheal epithelial cells and the bronchial epithelial cells are localized in a so-called ciliated pseudostratified columnar epithelium.

Thus, the ciliated pseudostratified columnar epithelium is found in the linings of the trachea as well as the upper respiratory tract. The pseudostratified epithelium can extend from the trachea to the distal bronchioles in human. For example, it can extend from 2° to 25 branches (large airways; tracheal epithelial cells) and from 2⁶ to 2²³ branches (small airways, bronchial epithelial cell). A pseudostratified epithelium appears to have multiple layers, but is actually only comprised of a single sheet of cells. The positioning of the nuclei within the individual columnar cells causes this illusion. These nuclei are found at various levels, creating a stratified appearance.

The major cell types of the tracheal and bronchial epithelium are ciliated cells, secretory cells and basal cells (Crystal et al. (2008) “Airway epithelial cells” Proc Am Thorac Soc, vol. 5, pp. 772-777; Rackley et al. (2012) “Building and maintaining the epithelium of the lung” The Journal of Clinical investigation, vol. 122, no. 8, pp. 2724-2730).

Consequently, it is envisioned that the lung epithelial cell may be a bronchial epithelial cell. It is further envisioned that the lung epithelial cell may be an alveolar epithelial cell. The bronchial or tracheal epithelial cell may be a ciliated cell, a secretory cell or a basal cell.

“Ciliated cells” as used herein have thin, tapering bases that are attached to the underlying basal lamina. The cells may also be attached to one another at their apical surfaces by tight junctions, forming a barrier physically impermeable to most substances, and laterally to one another and to basal cells by desmosomes. Intercellular spaces containing numerous microvilli may be present between the cells (Crystal et al. (2008) “Airway epithelial cells” Proc Am Thorac Soc, vol. 5, pp. 772-777; Rackley et al. (2012) “Building and maintaining the epithelium of the lung” The Journal of Clinical investigation, vol. 122, no. 8, pp. 2724-2730).

The secretory cell may be a Globlet cell, a Clara cell, a luminal secretory cell or a neuroendocrine cell.

A “Goblet cell” as used herein is located on the surface epithelium of upper and lower airways, and may produce mucus to coat the airways and trap particulates to be cleared.

“Clara cells” (nonciliated bronchiolar secretory cells) also called “club cells” as used herein line more distal airways and thus this cell type may be found primarily in membranous bronchioles. They are often columnar or (in the more distal airways) cuboidal in shape. They may secrete mature surfactant proteins A, B, D, and several detoxifying enzymes.

A “luminal secretory cell” as used herein is a non-ciliated cell. Luminal progenitor cells account for the majority of proliferating cells under resting conditions (Rackley et al. (2012) “Building and maintaining the epithelium of the lung” The Journal of Clinical investigation, vol. 122, no. 8, pp. 2724-2730).

Neuroendocrine cells may be attached at their bases to the basement membrane and may have tapering apices that extend toward and may or may not reach the airway surface. The principal function of the cells is the secretion of peptides (Crystal et al. (2008) “Airway epithelial cells” Proc Am Thorac Soc, vol. 5, pp. 772-777; Rackley et al. (2012) “Building and maintaining the epithelium of the lung” The Journal of Clinical investigation, vol. 122, no. 8, pp. 2724-2730).

“Basal cells” as used herein are a relatively abundant cell type that contacts the basement membrane, but may not contact the airway lumen. Basal cells can be located beneath the surface epithelium and serve as progenitors of both ciliated cells and secretory cells. They have a critical role in regeneration of the airway epithelium following injury. The expression of characteristic subsets of intermediate filament proteins (keratin 5 [K5], K6, K14, and K16) can distinguish basal cells from luminal epithelial cells (Rackley et al. (2012) “Building and maintaining the epithelium of the lung” The Journal of Clinical investigation, vol. 122, no. 8, pp. 2724-2730).

Thus, the lung epithelial cell as used herein can be a ciliated or non-ciliated tracheal epithelial cell. It is also contemplated that the lung epithelial cell as used herein can be a ciliated or non-ciliated bronchial epithelial cell.

The ciliated tracheal or bronchial cell may be a ciliated cell as described herein. The non-ciliated tracheal/bronchial cell may be a neuroendocrine cell, luminal secretory cell, Clara cell or basal cell.

The epithelial cell may be localized in a ciliated pseudostratified columnar epithelium or in a simple squamous epithelium. As described herein the tracheal and bronchial epithelial cells are included by the term “ciliated pseudostratified columnar epithelium”.

On the other hand, the simple squamous epithelium also referred to as alveolar epithelium herein is present after 2²³ branches (alveoli) and includes type I and type II cells. Thus, the alveolar epithelium comprises a mix of type I and II alveolar epithelium, or type I (AT1) and type II (AT2) alveolar epithelial cells. The alveoli are the smallest functional units in the respiratory tract, and are responsible for the exchange of gases such as oxygen and carbon dioxide with capillaries in the lungs. The alveolar epithelial monolayer is thin, consisting of squamous type I cells (AT1 that permit gas exchange) and cuboidal type 2 cells (AT2, that produce surfactant to enable lung expansion). Both cells transport ions and fluid from the alveolus to maintain dry airspaces.

AT1 cells cover ˜95% of the internal surface area of the lung. They are often branched cells with multiple apical surfaces that can extend into adjacent alveoli. The apical surface area of AT1 cells is large in comparison with most cells (i.e. ˜5,000 μm² for human AT1cells), yet they are very thin cells (i.e. 0.2 μm in depth). The gas exchange barrier is composed of AT1 and endothelial cells joined by fused basement membranes. Markers to determine if a cell is a type I cell are known to the skilled person and inter alia described by McElroy and Kasper (2004) “The use of alveolar epithelial type I cell-selective markers to investigate lung injury and repair” European Respiratory Journal 24: 664-673. For example, to determine if a cell is a type I cell the skilled person could perform immunohistochemistry to detect the expression of one or more of RTI₄₀/Tlα protein, HTIss and/or Na⁺/K⁺-ATPase α₂-isoform (α₂-isoform is depicted in SEQ ID NO. 5). The Na⁺/K⁺-ATPase α₂-isoform can have a sequence as shown in SEQ ID NO. 5 or be a sequence having 70%, 80%, 90%, 95%, 99% sequence identity to a sequence of SEQ ID NO. 5.

Additionally or alternatively, the skilled person could perform immunohistochemistry to detect the expression of one or more of advanced glycosylation end product-specific receptor (AGER, previously RAGE), podoplanin (PDPN, previously T1a), caveolin1 (CAV1), HOPX, GRAM domain 2 (GRAMD2) as inter alia described by Marconett et al. (2016) “Cross-Species Transcriptome Profiling Identifies New Alveolar Epithelial Type I Cell-Specific Genes” AJRCMB, vol 56, no. 3. The human GRAMD2 isoforms A and B are depicted in SEQ ID NO. 9 and 10. GRAMD2 can have a sequence as shown in SEQ ID NO. 9 or 10 or be a sequence having 70%, 80%, 90%, 95%, 99% sequence identity to a sequence of SEQ ID NO. 9 or 10.

Thus, the lung epithelial cell as described herein may be a cell that expresses the Na⁺/K⁺-ATPase α₂-isoform and/or GRAMD2. The lung epithelial cell may be a cell that expresses Na⁺/K⁺-ATPase α₂-isoform as shown in SEQ ID NO. 5 or be a sequence having 70%, 80%, 90%, 95%, 99% sequence identity to a sequence of SEQ ID NO. 5 and/or GRAMD2 as shown in SEQ ID NO. 9 or 10 or be a sequence having 70%, 80%, 90%, 95%, 99% sequence identity to a sequence of SEQ ID NO. 9 or 10.

AT2 are cuboidal cells situated between AT1 cells, and contain characteristic lamellar bodies and apical microvilli. AT2 cells have many known functions, including the production, secretion and reuptake of pulmonary surfactant and synthesis and secretion of immunomodulatory proteins important for host defence. Markers to determine if a cell is a type II cell are known to the skilled person and inter alia described by McElroy and Kasper (2004) “The use of alveolar epithelial type I cell-selective markers to investigate lung injury and repair” European Respiratory Journal 24: 664-673. For example, to determine if a cell is a type II cell the skilled person could perform immunohistochemistry to detect the expression of the pulmonary surfactant-associated protein C (SP-C). SP-C can have a sequence as shown in SEQ ID NO. 11 or be a sequence having 70%, 80%, 90%, 95%, 99% sequence identity to a sequence of SEQ ID NO. 11.

Thus, the lung epithelial cell as described herein may be a cell that expresses SP-C. The lung epithelial cell may be a cell that expresses SP-C as shown in SEQ ID NO. 11 or be a sequence having 70%, 80%, 90%, 95%, 99% sequence identity to a sequence of SEQ ID NO. 11.

Thus, the lung epithelial cell as described herein may also include cells that express SP-C and cells that express Na⁺/K⁺-ATPase α₂-isoform and/or GRAMD2.

Both, the type I and type II cell are known to the skilled person and inter alia described by Rackley et al. (2012) “Building and maintaining the epithelium of the lung” The Journal of Clinical investigation, vol. 122, no. 8, pp. 2724-2730, Crystal et al. (2008) “Airway epithelial cells” Proc Am Thorac Soc, vol. 5, pp. 772-777 and McElroy and Kasper (2004) “The use of alveolar epithelial type I cell-selective markers to investigate lung injury and repair” European Respiratory Journal 24: 664-673. Notably, the NKA as described herein is expressed in both AT1 and AT2 cells.

Thus, the lung epithelial cell can be an alveolar epithelial cell. The lung epithelial cell can also be a type I (AT1) and/or type II (AT2) cell.

The lung epithelial cell as disclosed herein can also be a lung epithelial cell, preferably an alveolar epithelial cell infected with a virus of the order Articulavirales, Mononegavirales, and/or Bunyavirales. It is further envisioned that lung epithelial cell, preferably an alveolar epithelial cell infected with a virus of the family Orthomyxoviridae (order Articulavirales), Arenaviridae, Hantaviridae, Mypoviridae, Nairovirdae, Peribunyaviridae, Phenuviridae (order Bunyavirales), Bornaviridae, Filoviridae, Paramyxoviridae and/or Sunviridae (order Mononegavirales).

It is also contemplated that the lung epithelial cell, preferably an alveolar epithelial cell is infected with a virus of the of the genus Alphainfluenzavirus, Betainfluenzavirus, Deltainfluenzavirus, Gammainfluenzavirus, preferably Alphainfluenzavirus (order Articulavirales; family Orthomyxoviridae), the subfamily Mammantavirinae, preferably the genus Loan virus, Mobat virus, Orthohantavirus or Thottimvirus (order Bunyavirales; family Hantavirididae), and/or the genus Pneumoviridae (order Mononegavirales, family Paramyoxoviridae).

It is further envisioned that the lung epithelial cell, preferably an alveolar epithelial cell infected with H1N1-, H1N2-, H2N2-, H3N2-, H5N1-, H6N1-, H7N2-, H7N3-, H7N7-, H7N9, H9N2-, H10N7-, H10N8- or H5N1-subtype (order Articulavirales; family Orthomyxoviridae, genus Alphainfluenzavirus), the Puumala virus, the Sin Nombre virus, the Seoul virus, the Hantaan virus, the Dobrava-Belgrad virus, the Saaremaa virus, Four corners virus or the Andes virus (order Bunyavirales; family Hantavirididae, subfamily Mammantavirinae, genus Orthohantavirus) or the Metapneumovirus, Orthopneumovirus such as Human orthopneumovirus like Human respiratory syncytial virus A, Human respiratory syncytial virus B or unclassified Human respiratory syncytial virus, Canine pneumovirus, Feline pneumovirus, Ovine respiratory syncytial virus, Ovine respiratory syncytial virus (strain WSU 83-1578), Pneumovirus, Respiratory syncytial virus, Swine pneumovirus or Pneumovirus sp (order Mononegavirales, family Paramyoxoviridae, genus Pneumoviridae).

It is also contemplated that the lung epithelial cell, preferably an alveolar epithelial cell is infected with 2 or more different viruses as disclosed herein.

The person skilled in the art knows how to detect if a lung epithelial cell is infected with a virus as disclosed herein. For example, the skilled person may take a tissue sample of the lung epithelial cell tissue. Then the skilled person may perform a PCR on this sample to analyze if viral genomic sequences or sequence fragments of the virus of interest are present in these cells.

A model for lung epithelial cells in general is the Calu3 cell line (human adenocarcinoma bronchial epithelial cells obtainable from American Type Culture Collection, Manassas, Va., USA) as used in the Examples and inter alia as described by Peteranderl et al. (2019) “Influenza A virus infection induces apical redistribution of Na⁺, K⁺-ATPase in lung epithelial cells in vitro and in vivo” American Journal of Respiratory Cell and Molecular Biology Volume 61 Number 3, pp. 395-397.

The ROCK inhibitor used herein can prevent apical NKA localization in lung epithelial cells. As disclosed herein the NKA is localized basolaterally within or associated to the plasma membrane of the polarized lung epithelial cells in healthy subjects. Basolateral localization can also be maintained during injury.

Notably, epithelial cells adhere to one another through tight junctions, desmosomes and adherens junctions, forming sheets of cells that line the surface of the animal/human body and internal cavities (e.g., respiratory tract, digestive tract and circulatory system). These cells have an apical-basal polarity defined by the apical membrane facing the outside surface of the body, or the lumen of internal cavities, and the basolateral membrane oriented away from the lumen. The basolateral membrane refers to both, the lateral membrane where cell-cell junctions connect neighboring cells and to the basal membrane where cells are attached to the basement membrane, a thin sheet of extracellular matrix proteins that separates the epithelial sheet from underlying cells and connective tissue (Wu and Mlodzik (2009). “A quest for the mechanism regulating global planar cell polarity of tissues”. Trends in Cell Biology. 19 (7): 295-305).

Thus, the term “basolateral”, when referred to a cell membrane, is the fraction of the plasma membrane, which faces adjacent cells and the underlying connective tissue. Similarly, the term “apical” referring to a cell membrane, is to mean the fraction of the cell membrane, which faces a lumen of a cavity.

FIG. 1 shows the effects of the ROCK inhibitor on NKA localisation. In healthy cells NKA is localized basolaterally. Upon viral infection NKA is localized basolaterally but also apically. The NKA distribution on the membrane of Calu 3 cells infected with Influenza A virus can be detected, for example, by optical visualization methods. For example, NKA can be visualized by means of antibody staining and confocal laser-scanning microscopy, as described in Example 7.

The present invention also envisiones that the ROCK inhibitor can reduce apical NKA localization in lung epithelial cells, compared to the apical localization before the administration of the ROCK inhibitor.

The inventors have surprisingly found that viral infection leads to apical NKA localization in lung epithelial cells in addition to the normally present basolateral NKA localization in lung epithelial cells. Upon administration of a ROCK inhibitor basolateral localization of the NKA localization in lung epithelial cells can be mostly restored, while the apical localization of the NKA localization in lung epithelial cells is reduced in comparison to the situation before administering the ROCK inhibitor.

Thus, the ROCK inhibitor results in a reduction of virally-induced apically localized NKA in lung epithelial cells of 5%, 10%, 15%, 20%, 30%, 40%, 50% 60%, 70%, 80%, 90% 95%, 99% or 100% compared to the apically localized NKA in lung epithelial cells present before the administration of the ROCK inhibitor.

The pulmonary edema as disclosed herein is associated with a virus infection, wherein the virus is of the order Articulavirales, Mononegavirales and/or Bunyavirales.

The virus of the order Articulavirales is preferably of the family Orthomyxoviridae.

The virus of the family Orthomyxoviridae can be of the genus Alphainfluenzavirus, Betainfluenzavirus, Deltainfluenzavirus, Gammainfluenzavirus, Isavirus, Quaranjavirus, Thogotovirus, unclassified Orthomyxoviridae.

The genus Alphainfluenzavirus may be a influenza A virus carrying any combination of hemagglutinin (HA) antigenic subtype (Hx) and neuraminidase (NA) antigenic subtype (N_{Y}). For example, the HA may be of sequence 12 or 13 or a sequence having sequence having 70%, 75%, 80%, 85%, 90%, 95%, 97%, 98%, 99% sequence identity to a sequence of SEQ ID NO. 11 or 12. Additionally or alternatively, the NA may be of sequence 14 or 15 or a sequence having sequence having 70%, 75%, 80%, 85%, 90%, 95%, 97%, 98%, 99% sequence identity to a sequence of SEQ ID NO. 14 or 15.

Non-limiting examples of alphainfluenzavirus include H1N1-, H1N2-, H2N2-, H3N2-, H5N1-, H6N1-, H7N2-, H7N3-, H7N7-, H7N9, H9N2-, H10N7-, H10N8- or H5N1-subtype. In one embodiment, the influenza A virus is of the H1N1-subtype. In other embodiments, the influenza A virus is of the H3N2-, H5N1- and H7N9-subtype. The influenza A virus can also be of the H3N2-, H5N1-, H1N1- and H7N9-subtype. The influenza A virus can also be the strain influenza virus A/Puerto Rico/8/34 (H1N1).

The virus may be of the order Bunyavirales. The skilled person knows which virus fall under the order Bunyavirales. All Bunyavirales have a negative-sense RNA genome, which is segmented into three parts.

The virus of the order Bunyavirales may be of the family Arenaviridae, Hantaviridae, Mypoviridae, Nairovirdae, Peribunyaviridae, or Phenuviridae.

The virus of the family Arenaviridae can be of the genus Mammarenavirus. Non-limiting examples of the genus mammarenavirus include inter alia the species Ippy-Virus (IPPYV), Lassa-Virus (LASV), Lujo-Virus (LUJV), Lunk-Virus (NKS-1), Lymphocytic choriomeningitis virus (LCMV), Mobala-Virus (MOBV), and Mopeia-Virus (Mopeia virus, MOPV).

The virus of the order Bunyavirales is preferably of the family Hantavirdae. The virus of the family Hantaviridae can of the subfamily Mammantavirinae. The virus of the subfamily Mammantavirinae may be of the genus Loan virus, Mobat virus, Orthohantavirus or Thottimvirus.

The genus Orthohantavirus inter alia includes the Puumala virus, the Sin Nombre virus, the Seoul virus, the Hantaan virus, the Dobrava-Belgrad virus, the Saaremaa virus, Four corners virus and the Andes virus.

The virus of the family Nairoviridae can be of the genus Orthonairovirus or Striwavirus. The orthonairovirus may be of the species Crimean-Congo hemorrhagic fever orthonairovirus (CCHFV).

The virus of the family Peribunyaviridae can be of the genus Orthobunyavirus or Pacuvirus.

The genus Orthobunyavirus can include the spezies Aino orthobunyavirus, Akabane orthobunyavirus, Anhembi orthobunyavirus, Anopheles B orthobunyavirus, Batai orthobunyavirus, Batama orthobunyavirus, Bertioga orthobunyavirus, Bunyamwera orthobunyavirus, Buttonwillow orthobunyavirus, Bwamba orthobunyavirus, Cache Valley orthobunyavirus, Cachoeira Porteira orthobunyavirus, Capim orthobunyavirus, Caraparu orthobunyavirus, Catu orthobunyavirus, Fort Sherman orthobunyavirus, Gamboa orthobunyavirus, Guama orthobunyavirus, Guaroa orthobunyavirus, laco orthobunyavirus, Ilesha orthobunyavirus, Ingwavuma orthobunyavirus, Jatobal orthobunyavirus, Kaeng Khoi orthobunyavirus, Keystone orthobunyavirus, La Crosse orthobunyavirus, Macaua orthobunyavirus, Madrid orthobunyavirus, Maguari orthobunyavirus, Manzanilla orthobunyavirus, Marituba orthobunyavirus, Mermet orthobunyavirus, Oriboca orthobunyavirus, Oropouche orthobunyavirus, Patois orthobunyavirus, Peaton orthobunyavirus, Sabo orthobunyavirus, Sango orthobunyavirus, Sathuperi orthobunyavirus, Schmallenberg orthobunyavirus, Shuni orthobunyavirus, Simbu orthobunyavirus, Snowshoe hare orthobunyavirus, Sororoca orthobunyavirus, Tahyna orthobunyavirus such as the Tahyna virus (TAHV), Tataguine orthobunyavirus, Tete orthobunyavirus, Utinga orthobunyavirus, Wolkberg orthobunyavirus, Wyeomyia orthobunyavirus, or Zegla orthobunyavirus. The genus Orthobunyavirus may also include the Baakal virus (BKAV).

The genus Pacuvirus can include the spezies Pacui virus (PACV), Rio Preto da Eva virus (RPEV) and Tapirape virus (TPPV).

The virus of the family Phenuiviridae is of the genus Banyangvirus, Goukovirus, or Phlebovirus.

The genus Phlebovirus may be of the species tick-borne Phlebovirus.

The virus of the order Mononegativirales may be of the family Bornaviridae, Filoviridae, Paramyxoviridae or Sunviridae. For example, the virus of the order Mononegativirales can be of the genus Pneumoviridae.

The virus of the family Bornaviridae can be of the genus Carbovirus, Orthobornavirus or unclassified Bornaviridae.

The virus of the family Filoviridae can be of the genus Cuevavirus, Ebolavirus or Marburgvirus.

The virus of the family Paramyxoviridae can be of the genus Avualvirinae, Avulavirus, Orthoparamyxovirinae, Rubulavirinae, Rubulavirus, unclassified Paramyyxoviridae, Metapneumovirus, Orthopneumovirus, or Pneumoviridae.

The virus of the genus Pneumoviridiae can be Metapneumovirus, Orthopneumovirus such as Human orthopneumovirus like Human respiratory syncytial virus A, Human respiratory syncytial virus B or unclassified Human respiratory syncytial virus, Canine pneumovirus, Feline pneumovirus, Ovine respiratory syncytial virus, Ovine respiratory syncytial virus (strain WSU 83-1578), Pneumovirus, Respiratory syncytial virus, Swine pneumovirus or Pneumovirus sp.

Thus, the virus as disclosed herein may be a virus of the family Orthomyxoviridae (order Articulavirales), Arenaviridae, Hantaviridae, Mypoviridae, Nairovirdae, Peribunyaviridae, Phenuviridae (order Bunyavirales), Bornaviridae, Filoviridae, Paramyxoviridae or Sunviridae (order Mononegavirales).

Thus, the virus as disclosed herein may be a virus of the genus Alphainfluenzavirus, Betainfluenzavirus, Deltainfluenzavirus, Gammainfluenzavirus, preferably Alphainfluenzavirus (order Articulavirales; family Orthomyxoviridae), the subfamily Mammantavirinae, preferably the genus Loan virus, Mobat virus, Orthohantavirus or Thottimvirus (order Bunyavirales; family Hantavirididae), or the genus Pneumoviridae (order Mononegavirales, family Paramyoxoviridae).

Thus, the virus as disclosed herein may be the H1N1-, H1N2-, H2N2-, H3N2-, H5N1-, H6N1-, H7N2-, H7N3-, H7N7-, H7N9, H9N2-, H10N7-, H10N8- or H5N1-subtype (order Articulavirales; family Orthomyxoviridae, genus Alphainfluenzavirus), the Puumala virus, the Sin Nombre virus, the Seoul virus, the Hantaan virus, the Dobrava-Belgrad virus, the Saaremaa virus, Four corners virus or the Andes virus (order Bunyavirales; family Hantavirididae, subfamily Mammantavirinae, genus Orthohantavirus) or the Metapneumovirus, Orthopneumovirus such as Human orthopneumovirus like Human respiratory syncytial virus A, Human respiratory syncytial virus B or unclassified Human respiratory syncytial virus, Canine pneumovirus, Feline pneumovirus, Ovine respiratory syncytial virus, Ovine respiratory syncytial virus (strain WSU 83-1578), Pneumovirus, Respiratory syncytial virus, Swine pneumovirus or Pneumovirus sp (order Mononegavirales, family Paramyoxoviridae, genus Pneumoviridae).

It is envisioned that the ROCK inhibitor reduces the virus load compared to the virus load before the administration of the ROCK inhibitor. For example, the viral load may be determined by measuring as plaque forming units (pfu)/ml.

In this context “reducing the viral load” may mean that viral particles, or infectious particles per mL are reduced by at least 10%, 20%, 30%, 40%, 50%, 60%, 70%, 80%, 90%, 95%, 99% or 100% compared to the infectious particles present before administration of the ROCK inhibitor.

It is further contemplated that the ROCK inhibitor can reduce the fluid weight of the lung compared to the fluid weight of the lung present before the administration of the ROCK inhibitor. How the fluid weight can be measured is described in the Examples.

It is also contemplated that the ROCK inhibitor reduces the infiltration of macrophages into the lung compared to the infiltration of macrophages into the lung before the administration of the ROCK inhibitor. How the infiltration of macrophages can be measured is described in the Examples.

The present invention also relates to the use of an in vitro test system comprising cultured lung epithelial cells infected with a virus of the order Articulavirales, Mononegatvirales and/or Bunyavirales, preferably an influenza virus, for the determination of inhibitors effective in preventing or reducing apical NKA localisation in lung epithelial cells. Preferably, the inhibitor is a ROCK inhibitor as described herein.

The inhibitor tested in the test system reduces apical NKA localisation in lung epithelial cells, when contacting the inhibitor with the in vitro test system compared to the apical NKA localization present in the in vitro test system before the contacting.

The in vitro test systems can be any suitable in vitro test system comprising cultured lung epithelial cells infected with a virus of the order Articulavirales, Mononegavirales and/or Bunyavirales, preferably an influenza virus. The influenza virus may be the strain influenza virus A/Puerto Rico/8/34 (H1N1). The cultured cells may be Calu-3 cells, cultured as e.g. described in Example 1.

The cultured lung epithelial cells may be human lung epithelial cells. The lung epithelial cells may be seeded at a density of about 1×10⁵, 2×10⁵, 3×10⁵, 4×10⁵, 5×10⁵, 6×10⁵, 7×10⁵, 8×10⁵, 9×10⁵, 10×10⁵, 11×10⁵ in medium.

The term “contacting” as used herein refers to the bringing virus-infected cultured lung epithelial cells spatially into close proximity to an inhibitor of interest. This can for example mean that an inhibitor of interest is applied to the medium in which the cultured cells are located via a syringe. As described herein, the step of contacting the cultured lung epithelial cells with a virus is carried out before the inhibitor is added to the cultured lung epithelial cells.

Thus, the use of an in vitro test system comprising cultured lung epithelial cells infected with an influenza virus can comprise contacting the test system with an inhibitor to be tested (inhibitor of interest).

If the contacting of the test system with the inhibitor results in a reduction of the apical localisation of NKA in lung epithelial cells compared to the apical localization of NKA present before contacting with the inhibitor, the inhibitor is efficient in reducing apical localisation of NKA in lung epithelial cells.

It is envisioned that the ROCK inhibitor reduces apical NKA localisation in lung epithelial cells, when contacting it with an in vitro test system, wherein the test system comprises cultured lung epithelial cells infected with a virus as described herein when compared to the in vitro test system before the contacting.

The present invention also relates to a composition comprising a ROCK inhibitor for use in a method for the prophylaxis and/or treatment of pulmonary edema by

i) preventing apical NKA localisation in lung epithelial cells, or
ii) reducing apical NKA localisation in lung epithelial cells compared to the apical NKA localisation present before the administration of the ROCK inhibitor,
wherein the pulmonary edema is associated with a virus infection, and wherein the virus is of the order Articulavirales, Mononegavirales and/or Bunyavirales.

The composition comprising a ROCK inhibitor may be a pharmaceutical composition. Preferably, such compositions further comprise a carrier, preferably a pharmaceutically acceptable carrier. The composition can be in the form of orally administrable suspensions or tablets; nasal sprays, sterile injectable preparations (intravenously, intrapleurally, intramuscularly), for example, as sterile injectable aqueous or oleaginous suspensions or suppositories. When administered orally as a suspension, these compositions are prepared according to techniques available in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art. The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The inhibitor or inhibitors are preferably administered in a therapeutically effective amount.

The present invention also relates to a method of treating a subject having or being at risk of pulmonary edema by preventing RNA virus associated apical NKA localisation in lung epithelial cells.

As used herein a “subject” can be any suitable subject. Preferably, the term “subject” as used herein refers to a mammal. The subject may be a dog, cat, horse, sheep, goat, cattle or a human subject, preferably a human subject. The subject may be a subject having pulmonary edema as described herein. The subject may be a subject infected with a virus of the order Articulavirus, Mononegavirales and/or Bunyavirales. Preferably, the subject is a subject infected with one or more virus of the Orthomyoxoviridae, Pneumoviridae and/or Hantaviridae. For example, it may be a subject infected with an influenza virus such as a influenza A virus.

As used herein a “subject at risk of infection” may be a subject at risk of developing a pulmonary edema as described herein. The subject may be at risk of infection with a virus of the order Articulavirus, Mononegavirales and/or Bunyavirales. Preferably, the subject is a subject at risk of infection with one or more virus of the Orthomyoxoviridae, Pneumoviridae and/or Hantaviridae. For example, it may be a subject at risk of infection with an influenza virus such as a influenza A virus.

It is further envisioned that the subject is a subject infected with or a subject at risk of infection with a virus of the family Orthomyxoviridae (order Articulavirales), Arenaviridae, Hantaviridae, Mypoviridae, Nairovirdae, Peribunyaviridae, Phenuviridae (order Bunyavirales), Bornaviridae, Filoviridae, Paramyxoviridae or Sunviridae (order Mononegavirales).

It is also contemplated that the subject is a subject infected with or a subject at risk of infection a virus of the of the genus Alphainfluenzavirus, Betainfluenzavirus, Deltainfluenzavirus, Gammainfluenzavirus, preferably Alphainfluenzavirus (order Articulavirales; family Orthomyxoviridae), the subfamily Mammantavirinae, preferably the genus Loan virus, Mobat virus, Orthohantavirus or Thottimvirus (order Bunyavirales; family Hantavirididae), or the genus Pneumoviridae (order Mononegavirales, family Paramyoxoviridae).

It is further envisioned that the subject is a subject infected with or a subject at risk of infection H1N1-, H1N2-, H2N2-, H3N2-, H5N1-, H6N1-, H7N2-, H7N3-, H7N7-, H7N9, H9N2-, H10N7-, H10N8- or H5N1-subtype (order Articulavirales; family Orthomyxoviridae, genus Alphainfluenzavirus), the Puumala virus, the Sin Nombre virus, the Seoul virus, the Hantaan virus, the Dobrava-Belgrad virus, the Saaremaa virus, Four corners virus or the Andes virus (order Bunyavirales; family Hantavirididae, subfamily Mammantavirinae, genus Orthohantavirus) or the Metapneumovirus, Orthopneumovirus such as Human orthopneumovirus like Human respiratory syncytial virus A, Human respiratory syncytial virus B or unclassified Human respiratory syncytial virus, Canine pneumovirus, Feline pneumovirus, Ovine respiratory syncytial virus, Ovine respiratory syncytial virus (strain WSU 83-1578), Pneumovirus, Respiratory syncytial virus, Swine pneumovirus or Pneumovirus sp (order Mononegavirales, family Paramyoxoviridae, genus Pneumoviridae).

It is also contemplated that the subject is a subject infected with 2 or more of the viruses as disclosed herein.

The present invention also relates to a method for detecting molecules effective in the prophylaxis and/or treatment of a pulmonary edema comprising contacting an in vitro test system comprising cultured lung epithelial cells infected with a virus of the order Articulavirales, Mononegavirales and/or Bunyavirales, preferably an influenza virus, with a compound of interest, wherein the compound of interest reduces apical NKA localisation in lung epithelial cells, compared to the in vitro test system before the contacting.

The compound of interest can be the ROCK inhibitor as described herein. Accordingly, the method can comprise the steps of (i) detecting the NKA cellular localization on the lung epithelial cells infected with a virus of the order Articulavirales, Mononegavirales and/or Bunyavirales, preferably an influenza virus in the in vitro test system as described herein (ii) contacting the in vitro test system with a compound of interest, (iii) detecting the NKA cellular localization on the lung epithelial cells after contacting the in vitro test system with the compound of interest of step (iii) and (iv) analyzing localization (i.e migration) of the NKA in the in vitro test system after the contacting, wherein a decrease of apical NKA localization compared to the apical NKA localization before contacting indicates that the inhibitor is effective in the prophylaxis and/or treatment of pulmonary edema.

The present invention also relates to a test system comprising

i) a ROCK inhibitor;
ii) lung epithelial cells;
iii) a virus of the order Articulavirales, Mononegavirales and/or Bunyavirales; and
iv) means for the detection and cellular localization of NKA.
As used herein “means for the detection and cellular localization of NKA” can be any suitable mean. Such means are known to the skilled person. For example, the skilled person may be used the means as described in the Examples.

The following sequences have been referred to by in the present disclosure.

| SEQ | What | Sequence |
|---|---|---|
| 1 | Uniprot no. | MARGKAKEEGSWKKFIWNSEKKEFLGRTGGSWFKILLFYVIFYGCLAGIFIG |
| | P05026 | TIQVMLLTISEFKPTYQDRVAPPGLTQIPQIQKTEISFRPNDPKSYEAYVLNIV |
| | Sodium/ | RFLEKYKDSAQRDDMIFEDCGDVPSEPKERGDFNHERGERKVCRFKLEW |
| | potassium- | LGNCSGLNDETYGYKEGKPCIIIKLNRVLGFKPKPPKNESLETYPVMKYNPN |
| | transporting | VLPVQCTGKRDEDKDKVGNVEYFGLGNSPGFPLQYYPYYGKLLQPKYLQP |
| | ATPase subunit | LLAVQFTNLTMDTEIRIECKAYGENIGYSEKDRFQGRFDVKIEVKS |
| | beta-1 | |
| | Homo sapiens | |
| 2 | Uniprot no. | MVIQKEKKSCGQVVEEWKEFVWNPRTHQFMGRTGTSWAFILLFYLVFYGF |
| | P14415 | LTAMFTLTMWVMLQTVSDHTPKYQDRLATPGLMIRPKTENLDVIVNVSDTE |
| | Sodium/ | SWDQHVQKLNKFLEPYNDSIQAQKNDVCRPGRYYEQPDNGVLNYPKRAC |
| | potassium- | QFNRTQLGNCSGIGDSTHYGYSTGQPCVFIKMNRVINFYAGANQSMNVTC |
| | transporting | AGKRDEDAENLGNFVMFPANGNIDLMYFPYYGKKFHVNYTQPLVAVKFLN |
| | ATPase subunit | VTPNVEVNVECRINAANIATDDERDKFAGRVAFKLRINKT |
| | beta-2 | |
| | Homo sapiens | |
| 3 | Uniprot no. | MTKNEKKSLNQSLAEWKLFIYNPTTGEFLGRTAKSWGLILLFYLVFYGFLAA |
| | P54709 | LFSFTMWVMLQTLNDEVPKYRDQIPSPGLMVFPKPVTALEYTFSRSDPTSY |
| | Sodium/ | AGYIEDLKKFLKPYTLEEQKNLTVCPDGALFEQKGPVYVACQFPISLLQACS |
| | potassium- | GMNDPDFGYSQGNPCILVKMNRIIGLKPEGVPRIDCVSKNEDIPNVAVYPH |
| | transporting | NGMIDLKYFPYYGKKLHVGYLQPLVAVQVSFAPNNTGKEVTVECKIDGSAN |
| | ATPase subunit | LKSQDDRDKFLGRVMFKITARA |
| | beta-3 | |
| | Homo sapiens | |
| 4 | Uniprot no. | MGKGVGRDKYEPAAVSEQGDKKGKKGKKDRDMDELKKEVSMDDHKLSL |
| | P05023 | DELHRKYGTDLSRGLTSARAAEILARDGPNALTPPPTTPEWIKFCRQLFGG |
| | Sodium/ | FSMLLWIGAILCFLAYSIQAATEEEPQNDNLYLGVVLSAVVIITGCFSYYQEA |
| | potassium- | KSSKIMESFKNMVPQQALVIRNGEKMSINAEEVVVGDLVEVKGGDRIPADL |
| | transporting | RIISANGCKVDNSSLTGESEPQTRSPDFTNENPLETRNIAFFSTNCVEGTAR |
| | ATPase subunit | GIVVYTGDRTVMGRIATLASGLEGGQTPIAAEIEHFIHIITGVAVFLGVSFFILS |
| | alpha-1 | LILEYTWLEAVIFLIGIIVANVPEGLLATVTVCLTLTAKRMARKNCLVKNLEAV |
| | Homo sapiens | ETLGSTSTICSDKTGTLTQNRMTVAHMWFDNQIHEADTTENQSGVSFDKT |
| | | SATWLALSRIAGLCNRAVFQANQENLPILKRAVAGDASESALLKCIELCCGS |
| | | VKEMRERYAKIVEIPFNSTNKYQLSIHKNPNTSEPQHLLVMKGAPERILDRC |
| | | SSILLHGKEQPLDEELKDAFQNAYLELGGLGERVLGFCHLFLPDEQFPEGF |
| | | QFDTDDVNFPIDNLCFVGLISMIDPPRAAVPDAVGKCRSAGIKVIMVTGDHP |
| | | ITAKAIAKGVGIISEGNETVEDIAARLNIPVSQVNPRDAKACVVHGSDLKDMT |
| | | SEQLDDILKYHTEIVFARTSPQQKLIIVEGCQRQGAIVAVTGDGVNDSPALK |
| | | KADIGVAMGIAGSDVSKQAADMILLDDNFASIVTGVEEGRLIFDNLKKSIAYT |
| | | LTSNIPEITPFLIFIIANIPLPLGTVTILCIDLGTDMVPAISLAYEQAESDIMKRQ |
| | | PRNPKTDKLVNERLISMAYGQIGMIQALGGFFTYFVILAENGFLPIHLLGLRV |
| | | DWDDRWINDVEDSYGQQWTYEQRKIVEFTCHTAFFVSIVVVQWADLVICK |
| | | TRRNSVFQQGMKNKILIFGLFEETALAAFLSYCPGMGVALRMYPLKPTWW |
| | | FCAFPYSLLIFVYDEVRKLIIRRRPGGWVEKETYY |
| 5 | Uniprot no. | MGRGAGREYSPAATTAENGGGKKKQKEKELDELKKEVAMDDHKLSLDEL |
| | P50993 | GRKYQVDLSKGLTNQRAQDVLARDGPNALTPPPTTPEWVKFCRQLFGGF |
| | Sodium/ | SILLWIGAILCFLAYGIQAAMEDEPSNDNLYLGVVLAAVVIVTGCFSYYQEAK |
| | potassium- | SSKIMDSFKNMVPQQALVIREGEKMQINAEEVVVGDLVEVKGGDRVPADL |
| | transporting | RIISSHGCKVDNSSLTGESEPQTRSPEFTHENPLETRNICFFSTNCVEGTAR |
| | ATPase subunit | GIVIATGDRTVMGRIATLASGLEVGRTPIAMEIEHFIQLITGVAVFLGVSFFVL |
| | alpha-2 | SLILGYSWLEAVIFLIGIIVANVPEGLLATVTVCLTLTAKRMARKNCLVKNLEA |
| | | VETLGSTSTICSDKTGTLTQNRMTVAHMWFDNQIHEADTTEDQSGATFDK |
| | | RSPTWTALSRIAGLCNRAVFKAGQENISVSKRDTAGDASESALLKCIELSC |
| | | GSVRKMRDRNPKVAEIPFNSTNKYQLSIHEREDSPQSHVLVMKGAPERILD |
| | | RCSTILVQGKEIPLDKEMQDAFQNAYM |
| | | ELGGLGERVLGFCQLNLPSGKFPRGFKFDTDELNFPTEKLCFVGLMSMIDP |
| | | PRAAVPDAVGKCRSAGIKVIMVTGDHPITAKAIAKGVGIISEGNETVEDIAAR |
| | | LNIPMSQVNPREAKACVVHGSDLKDMTSEQLDEILKNHTEIVFARTSPQQK |
| | | LIIVEGCQRQGAIVAVTGDGVNDSPALKKADIGIAMGISGSDVSKQAADMILL |
| | | DDNFASIVTGVEEGRLIFDNLKKSIAYTLTSNIPEITPFLLFIIANIPLPLGTVTIL |
| | | CIDLGTDMVPAISLAYEAAESDIMKRQPRNSQTDKLVNERLISMAYGQIGMI |
| | | QALGGFFTYFVILAENGFLPSRLLGIRLDWDDRTMNDLEDSYGQEWTYEQ |
| | | RKVVEFTCHTAFFASIVVVQWADLIICKTRRNSVFQQGMKNKILIFGLLEETA |
| | | LAAFLSYCPGMGVALRMYPLKVTWWFCAFPYSLLIFIYDEVRKLILRRYPGG |
| | | WWEKETYY |
| 6 | Uniprot no. | MGDKKDDKDSPKKNKGKERRDLDDLKKEVAMTEHKMSVEEVCRKYNTDC |
| | P13637 | VQGLTHSKAQEILARDGPNALTPPPTTPEWVKFCRQLFGGFSILLWIGAILC |
| | Sodium/ | FLAYGIQAGTEDDPSGDNLYLGIVLAAVVIITGCFSYYQEAKSSKIMESFKN |
| | potassium- | MVPQQALVIREGEKMQVNAEEVVVGDLVEIKGGDRVPADLRIISAHGCKVD |
| | transporting | NSSLTGESEPQTRSPDCTHDNPLETRNITFFSTNCVEGTARGVVVATGDRT |
| | ATPase subunit | VMGRIATLASGLEVGKTPIAIEIEHFIQLITGVAVFLGVSFFILSLILGYTWLEA |
| | alpha-3 | VIFLIGIIVANVPEGLLATVTVCLTLTAKRMARKNCLVKNLEAVETLGSTSTIC |
| | | SDKTGTLTQNRMTVAHMWFDNQIHEADTTEDQSGTSFDKSSHTWVALSHI |
| | | AGLCNRAVFKGGQDNIPVLKRDVAGDASESALLKCIELSSGSVKLMRERNK |
| | | KVAEIPFNSTNKYQLSIHETEDPNDNRYLLVMKGAPERILDRCSTILLQGKE |
| | | QPLDEEMKEAFQNAYLELGGLGERVLGFCHYYLPEEQFPKGFAFDCDDVN |
| | | FTTDNLCFVGLMSMIDPPRAAVPDAVGKCRSAGIKVIMVTGDHPITAKAIAK |
| | | GVGIISEGNETVEDIAARLNIPVSQVNPRDAKACVIHGTDLKDFTSEQIDEIL |
| | | QNHTEIVFARTSPQQKLIIVEGCQRQGAIVAVTGDGVNDSPALKKADIGVAM |
| | | GIAGSDVSKQAADMILLDDNFASIVTGVEEGRLIFDNLKKSIAYTLTSNIPEIT |
| | | PFLLFIMANIPLPLGTITILCIDLGTDMVPAISLAYEAAESDIMKRQPRNPRTD |
| | | KLVNERLISMAYGQIGMIQALGGFFSYFVILAENGFLPGNLVGIRLNWDDRT |
| | | VNDLEDSYGQQWTYEQRKVVEFTCHTAFFVSIVVVQWADLIICKTRRNSVF |
| | | QQGMKNKILIFGLFEETALAAFLSYCPGMDVALRMYPLKPSWWFCAFPYSF |
| | | LIFVYDEIRKLILRRNPGGWVEKETYY |
| 7 | Uniprot no. | MGLWGKKGTVAPHDQSPRRRPKKGLIKKKMVKREKQKRNMEELKKEVVM |
| | Q13733 | DDHKLTLEELSTKYSVDLTKGHSHQRAKEILTRGGPNTVTPPPTTPEWVKF |
| | Sodium/ | CKQLFGGFSLLLWTGAILCFVAYSIQIYFNEEPTKDNLYLSIVLSVVVIVTGCF |
| | potassium- | SYYQEAKSSKIMESFKNMVPQQALVIRGGEKMQINVQEVVLGDLVEIKGGD |
| | transporting | RVPADLRLISAQGCKVDNSSLTGESEPQSRSPDFTHENPLETRNICFFSTN |
| | ATPase subunit | CVEGTARGIVIATGDSTVMGRIASLTSGLAVGQTPIAAEIEHFIHLITVVAVFL |
| | alpha-4 | GVTFFALSLLLGYGWLEAIIFLIGIIVANVPEGLLATVTVCLTLTAKRMARKNC |
| | Homo sapiens | LVKNLEAVETLGSTSTICSDKTGTLTQNRMTVAHMWFDMTVYEADTTEEQ |
| | | TGKTFTKSSDTWFMLARIAGLCNRADFKANQEILPIAKRATTGDASESALLK |
| | | FIEQSYSSVAEMREKNPKVAEIPFNSTNKYQMSIHLREDSSQTHVLMMKGA |
| | | PERILEFCSTFLLNGQEYSMNDEMKEAFQNAYLELGGLGERVLGFCFLNLP |
| | | SSFSKGFPFNTDEINFPMDNLCFVGLISMIDPPRAAVPDAVSKCRSAGIKVI |
| | | MVTGDHPITAKAIAKGVGIISEGTETAEEVAARLKIPISKVDASAAKAIVVHGA |
| | | ELKDIQSKQLDQILQNHPEIVFARTSPQQKLIIVEGCQRLGAVVAVTGDGVN |
| | | DSPALKKADIGIAMGISGSDVSKQAADMILLDDNFASIVTGVEEGRLIFDNLK |
| | | KSIMYTLTSNIPEITPFLMFIILGIPLPLGTITILCIDLGTDMVPAISLAYESAESD |
| | | IMKRLPRNPKTDNLVNHRLIGMAYGQIGMIQALAGFFTYFVILAENGFRPVD |
| | | LLGIRLHWEDKYLNDLEDSYGQQWTYEQRKVVEFTCQTAFFVTIVVVQWA |
| | | DLIISKTRRNSLFQQGMRNKVLIFGILEETLLAAFLSYTPGMDVALRMYPLKI |
| | | TWWLCAIPYSILIFVYDEIRKLLIRQHPDGWVERETYY |
| 8 | Uniprot no. | MGDWSALGKLLDKVQAYSTAGGKVWLSVLFIFRILLLGTAVESAWGDEQS |
| | P17302 | AFRCNTQQPGCENVCYDKSFPISHVRFWVLQIIFVSVPTLLYLAHVFYVMR |
| | Gap junction | KEEKLNKKEEELKVAQTDGVNVDMHLKQIEIKKFKYGIEEHGKVKMRGGLL |
| | alpha-1 | RTYHSILFKSIFEVAFLLIQWYIYGFSLSAVYTCKRDPCPHQVDCFLSRPTEK |
| | protein | TIFIIFMLVVSLVSLALNIIELFYVFFKGVKDRVKGKSDPYHATSGALSPAKDC |
| | Homo sapiens | GSQKYAYFNGCSSPTAPLSPMSPPGYKLVTGDRNNSSCRNYNKQASEQN |
| | | WANYSAEQNRMGQAGSTISNSHAQPFDFPDDNQNSKKLAAGHELQPLAIV |
| | | DQRPSSRASSRASSRPRPDDLEI |
| 9 | Uniprot no. | MTALSRSEATEEGGNQQMHRKTASLNSPVSCKEKPDRVEEPPDYSLHWP |
| | Q8IUY3 | EGLKGEEIKKCGREGITLNKYNQQYHKLFKDVPLEEVVLKVCSCALQRDFL |
| | HUMAN GRAM | LQGRLYISPNWLCFHASLFGKDIKVVIPVVSVQMIKKHKMARLLPNGLAITTN |
| | domain- | TSQKYIFVSLLSRDSVYDLLRRVCTHLQPSSKKSLSVREFSGEPESLEVLIP |
| | containing | EMKWRKVCPSSRSLSLPDNIPCIPPSSVDSTDSFFPSRKPPMSEKSRAQV |
| | protein 2A | ASENGGRWAWPMPGWGPACPKKMPNCSPTAKNAVYEEDELEEEPRSTG |
| | (GRAMD2A) | ELRLWDYRLLKVFFVLICFLVMSSSYLAFRISRLEQQLCSLSWDDPVPGHR |
| 10 | Uniprot no. | MTELQQDVEDTKPAKVLGKRESKLGSAHSEAENGVEEKKKACRSPTAQSP |
| | Q96HH9\|GRM2 | TPSVEADSPDQKKIISLWSKSSFDGASLASDKNDCKTESKNDPKTERKKSS |
| | B_HUMAN | SSSQYKANMHFHKLFLSVPTEEPLKQSFTCALQKEILYQGKLFVSENWICF |
| | GRAM domain- | HSKVFGKDTKISIPAFSVTLIKKTKTALLVPNALIIATVTDRYIFVSLLSRDSTY |
| | containing | KLLKSVCGHLENTSVGNSPNPSSAENSFRADRPSSLPLDFNDEFSDLDGV |
| | protein 2B | VQQRRQDMEGYSSSGSQTPESENSRDFHATESQTVLNVSKGEAKPTRAD |
| | (GRAMD2B) | AHVNRVPEGKAKSLPVQGLSETVGILHKVKSQKCPMLHHILIFYAIVVCALIIS |
| | | TFYMRYRINTLEEQLGLLTSIVDTHNTEQAAPSGLRSQVQFNVEVLCQELT |
| | | ANIVKLEKIQNNLQKLLENGD |
| 11 | Uniprot no. | MDVGSKEVLMESPPDYSAAPRGRFGIPCCPVHLKRLLIVVVVVVLIVVVIVG |
| | P11686 | ALLMGLHMSQKHTEMVLEMSIGAPEAQQRLALSEHLVTTATFSIGSTGLVV |
| | HUMAN | YDYQQLLIAYKPAPGTCCYIMKIAPESIPSLEALNRKVHNFQMECSLQAKPA |
| | Pulmonary | VPTSKLGQAEGRDAGSAPSGGDPAFLGMAVNTLCGEVPLYYI |
| | surfactant- | |
| | associated | |
| | protein C | |
| | (SP-C) | |
| 12 | Uniprot no. | MAIIYLILLFTAVRGDQICIGYHANNSTEKVDTNLERNVTVTHAKDILEKTHNG |
| | P03451 | KLCKLNGIPPLELGDCSIAGWLLGNPECDRLLSVPEWSYIMEKENPRDGLC |
| | HEMA_I57A0 | YPGSFNDYEELKHLLSSVKHFEKVKILPKDRWTQHTTTGGSRACAVSGNP |
| | Hemagglutinin | SFFRNMVWLTKEGSDYPVAKGSYNNTSGEQMLIIWGVHHPIDETEQRTLY |
| | Influenza A | QNVGTYVSVGTSTLNKRSTPEIATRPKVNGQGGRMEFSWTLLDMWDTINF |
| | virus (strain | ESTGNLIAPEYGFKISKRGSSGIMKTEGTLENCETKCQTPLGAINTTLPFHN |
| | A/Japan/305/ | VHPLTIGECPKYVKSEKLVLATGLRNVPQIESRGLFGAIAGFIEGGWQGMV |
| | 1957 H2N2) | DGWYGYHHSNDQGSGYAADKESTQKAFDGITNKVNSVIEKMNTQFEAVG |
| | | KEFGNLERRLENLNKRMEDGFLDVWTYNAELLVLMENERTLDFHDSNVKN |
| | | LYDKVRMQLRDNVKELGNGCFEFYHKCDDECMNSVKNGTYDYPKYEEES |
| | | KLNRNEIKGVKLSSMGVYQILAIYATVAGSLSLAIMMAGISFWMCSNGSLQC |
| | | RICI |
| 13 | Uniprot no. | MKTIIALSYIFCLALGQDLPGNDNNTATLCLGHHAVPNGTLVKTITDDQIEVT |
| | P03436 | NATELVQSSSTGKICNNPHRILDGIDCTLIDALLGDPHCDVFQNETWDLFVE |
| | HEMA_I68A6 | RSKAFSNCYPYDVPDYASLRSLVASSGTLEFITEGFTWTGVTQNGGSNAC |
| | Hemagglutinin | KRGPDSGFFSRLNWLTKSGSTYPVLNVTMPNNDNFDKLYIWGVHHPSTNQ |
| | Influenza A | EQTSLYVQASGRVTVSTRRSQQTIIPNIGSRPWVRGQSSRISIYWTIVKPGD |
| | virus (strain | VLVINSNGNLIAPRGYFKMRTGKSSIMRSDAPIDTCISECITPNGSIPNDKPF |
| | A/Northern | QNVNKITYGACPKYVKQNTLKLATGMRNVPEKQTRGLFGAIAGFIENGWE |
| | Territory/60/ | GMIDGWYGFRHQNSEGTGQAADLKSTQAAIDQINGKLNRVIEKTNEKFHQI |
| | 1968 H3N2) | EKEFSEVEGRIQDLEKYVEDTKIDLWSYNAELLVALENQHTIDLTDSEMNKL |
| | | FEKTRRQLRENAEDMGNGCFKIYHKCDNACIESIRNGTYDHDVYRDEALN |
| | | NRFQIKGVELKSGYKDWILWISFAISCFLLCVVLLGFIMWACQRGNIRCNICI |
| 14 | Uniprot no. | MNPNQKIITIGSVSLTIATVCFLMQIAILATTVTLHFKQHKCDSPASNQVMPC |
| | Q1K9Q1 | EPIIIERNITEIVYLNNTTIEKEICPEVVEYRNWSKPQCQITGFAPFSKDNSIRL |
| | NRAM_I57A0 | SAGGDIWVTREPYVSCDPGKCYQFALGQGTTLDNKHSNGTIHDRIPHRTLL |
| | Neuraminidase | MNELGVPFHLGTKQVCVAWSSSSCHDGKAWLHVCVTGDDRNATASFIYD |
| | OS = Influenza | GRLVDSIGSWSQNILRTQESECVCINGTCTVVMTDGSASGRADTRILFIKEG |
| | A virus | KIVHIGPLSGSAQHIEECSCYPRYPDVRCICRDNWKGSNRPVIDINMEDYSI |
| | (strain | DSSYVCSGLVGDTPRNDDSSSNSNCRDPNNERGNPGVKGWAFDNGDDV |
| | A/Japan/305/ | WMGRTISKDLRSGYETFKVIGGWSTPNSKSQVNRQVIVDNNNWSGYSGIF |
| | 1957 H2N2) | SVEGKSCINRCFYVELIRGRPQETRVWWTSNSIVVFCGTSGTYGTGSWPD |
| | | GANINFMPI |
| 15 | Uniprot no. | MNPNQKIITIGSVSLTIATVCFLMQTAILVTTVTLHFKQYECDSPASNQVMPC |
| | P03473 | EPIIIERNITEIVYLNNTTIEKEICPKVVEYRNWSKPQCQITGFAPFSKDNSIRL |
| | NRAM_I68A6 | SAGGDIWVTREPYVSCDHGKCYQFALGQGTTLDNKHSNDTIHDRIPHRTLL |
| | Neuraminidase | MNELGVPFHLGTRQVCIAWSSSSCHDGKAWLHVCITGDDKNATASFIYDG |
| | Influenza A | RLVDSIGSWSQNILRTQESECVCINGTCTVVMTDGSASGRADTRILFIEEGK |
| | virus (strain | IVHISPLSGSAQHVEECSCYPRYPGVRCICRDNWKGSNRPVVDINMEDYSI |
| | A/Northern | DSSYVCSGLVGDTPRNDDRSSNSNCRNPNNERGNQGVKGWAFDNGDDV |
| | Territory/60/ | WMGRTISKDLRSGYETFKVIGGWSTPNSKSQINRQVIVDSDNRSGYSGIFS |
| | 1968 H3N2) | VEGKSCINRCFYVELIRGRKQEARVWWTSNSIVVFCGTSGTYGTGSWPDG |
| | | ANINFMPI |

### EXAMPLES

The following examples illustrate the invention. These examples should not be construed as to limit the scope of this invention. The examples are included for purposes of illustration and the present invention is limited only by the claims.

| Cell culture | Origin | Source | Media composition |
|---|---|---|---|
| Calu3 | human | American Type Culture | MEM, 15% hi FCS, |
| | adenocarcinoma | Collection, Manassas, VA, | 4 mM GlutaMax, 1 mM |
| | bronchial epithelial | USA | Sodium Pyruvate, 1% |
| | cells | | Non-Essential Amino |
| | | | Acids Solution |
| MDCK II | canine kidney | Cell culture collection, | DMEM, 10% hi FCS, |
| | epithelial cells, | Institute of Medical Virology, | 25 mM Glucose, 4 mM L- |
| | subclone II. | Justus-Liebig University | Glutamine |
| | | Giessen, Germany | |

### Example 1—Cell Culture Cultivation

All cell lines were cultivated in 75 cm² or 165 cm² tissue culture flask at 37° C. in a 95% humidified atmosphere of 5% CO₂. When cell monolayers reached 90% confluence cells (except Calu3 cells which do not reach 100% confluence, maximum 50%) were washed once with PBS −/− and detached with Trypsin-EDTA. Cells were resuspended in a suitable media (see table above) and seeded in 6-well, 12-well, 24-well tissue plates, in 15 cm tissue dishes or on sterile glass-cover slips placed within a culture well 24 hours prior of each experiment.

### Example 2—Polarization of Calu3 Cells

In order to obtain highly polarized Calu3 cells, the cell monolayer in a 175 cm² tissue culture flask grown to 50% confluence was washed once with PBS −/− and treated with Trypsin-EDTA. Cells were resuspended in 10 ml culture medium and centrifuged for 15 min at 300×g, 24° C. Supernatant was discarded and cells were resuspended in 5 ml of culture medium. 30 μl of cell suspension were mixed with 30 μl of 0.4% trypan blue dye and cell concentration was calculated in Neubauer chamber according manufacturer's instruction. Cells were dilute to a concentration of 2×10⁶ viable cells/ml in Calu3 culture medium and 250 μl/125 μl of the cell suspension containing 0.5×10⁶/0.25×10⁶ viable cells were placed to the apical compartment of each Transwells® insert in a 12/24 well plate. 1/0.3 ml of Calu-3 culture medium was added into the basolateral compartments, avoiding the introduction of air bubbles and cells were cultivated at 37° C., 5% CO₂. For the cells grown under the Liquid-Liquid Interface (LLI) condition, medium was replaced in both compartments each second day. For Air-Liquid Interface (ALI), culture medium was aspirated from the apical compartment on the day two, whereas medium was replaced every 2 days in the basolateral compartments.

### Example 3—Cell Viability Assay

In order to check a cytotoxicity of applied inhibitors a commercial available reagent PrestoBlue™ has been used. The reagent-resazurin (7-hydroxy-10-oxidophenoxazin-10-ium-3-one)-based compound is converted into the reduced form by the mitochondrial enzymes of viable cells with a change of color and can be quantified using either spectrophotometric or fluorometric approach. The viability assay was performed according to the manufacturer's protocol. Calu3 cells were seeded on a 96-well plate in a concentration 1×10⁴/well in 90 μl of culture medium 24 h later were treated with media containing the inhibitors at different concentrations and 24 h later 10 μl of 10-fold ready-to-use PrestoBlue™ reagent were added to each well. The plate was then incubated 30 min at 37° C. in darkness and subsequently the absorbance was measured at 570 nm wavelength by Tecan Spark® 10M multimode microplate reader to determine the amount of resazurin conversion.

### Example 4—Vectorial Water Transport

Vectorial water transport (VWT) as a characteristic of the physiological status of the Calu3 cell monolayer was measured by changes of FITC-dextran concentrations in apical and basal cell culture medium of polarized Calu3 cells grown on Transwells® inserts for 14 days under Liquid-Liquid Interface conditions. For this, the cells were either mock infected or infected with PR8 at a multiplicity of infection (MOI): 2 for 1 hour at 37° C. Inoculum was removed and cells were supplied with the Infection medium #2 containing 1 mg/ml of 70 kDa FITC-dextran and 5 μM Rho-kinase inhibitor XIII in DMSO or just the equal amount of DMSO (solvent). Cells were incubated at 37° C. for 8 and 24 h. 30 μl of cell culture medium from apical and basal side were collected, diluted 1:1 with PBS −/− and placed on 96 well flat bottom black plates. The fluorescence intensity of the samples was measured at excitation wavelength 480 nm and emission wavelength 535 nm by Tecan Spark® 10M multimode microplate reader. VWT was calculated using the formula:

C0=[1−(C0/Ca)]−[1−(C/Cb)], where

C0—fluorescence value of culture medium at starting point;
Ca—fluorescence value of culture medium in apical side of Transwells® inserts;
Cb—fluorescence value of culture medium in basal side of Transwells® inserts.
To determine whether the NKA misdistribution during IAV infection is a general characteristic of IAV pathogenicity, different IAV subtypes were screened for their ability to induce an apical NKA presentation. Firstly, the growth kinetics of influenza virus A/Victoria/3/75 (H3N2), A/Thailand/1 (KAN-1)/2004 (H5N1) or A/Anhui/1/2013 (H7N9) in Calu3 cells were compared. No significant differences in the replication efficiency of the analyzed IAV subtypes and the previously tested PR8 virus were detected. Highly pathogenic viruses of the H5N1- and H7N9 subtype demonstrated maximal virus titer equal to 6.1 log 10 FFU/ml and 7.5 log 10 FFU/ml, respectively, 48 h p.i., whereas the less pathogenic H3N2 strain reached a maximal titer of 6.9 log 10 FFU/ml 24 h p.i. (FIG. 11A). All tested viruses were able to induce apical NKA mislocalization at the late stage of viral infection as demonstrated by OCWB analysis (FIG. 11B). There was no significant difference in the capacity to cause apical NKA appearance between the different IAV strains, indicating that this is general effect caused by IAV infection of polarized epithelia cells.

### Example 5—Virus Propagation

The influenza virus A/Puerto Rico/8/34 (H1N1) was propagated in MDCK II cells in 165 cm² culture flask. For this a 24-hr-old 85% confluent monolayer of cells was washed once with PBS −/− and 5 ml of PBS+/+/BA/PS containing virus dilution corresponding to MOI equal to 0.01 were added, followed by 45 min of incubation at a room temperature. Subsequently, inoculum was removed, cells were washed with PBS −/− and were incubated in Infection medium #1 containing 1 mg TPCK-treated trypsin ml⁻¹ at 37° C. for 2 days. Supernatant was collected and virus titer was determined by a foci-forming assay.

Foci Forming Assay

For the foci forming assay, MDCK II cells were seeded in 96-well plates at a concentration of 3×10⁶ cells/plate. The next day, 10-fold dilutions in duplicates (from 10⁻¹ to 10⁻⁸) in PBS+/+/BA/PS was prepared from each virus sample in U-shaped 96-well. Importantly, during the preparation of the dilutions the pipet tips were changed after each dilution step. The MDCK II cells in the 96-well plate(s) were washed once with PBS+/+. Then 50 μl of the according dilutions for each sample in the U-shaped plate were transferred onto the MDCK II cells in an according well of the 96-well plate, which were then incubated for 45 min. After incubation, inoculum was removed starting with the 10⁻⁸ dilution row without changing the pipet tips and 100 μl of Avicel medium containing 1 mg TPCK-treated trypsin ml⁻¹ were added to each well. Cells were incubated at 37° C., 5% CO₂ for 30 hours followed by the immunocytochemical analysis to detect virus-infected cells. For this, cells were washed twice with 200 μl of PBS+/+, were fixed and permeabilized in 4% (w/v) paraformaldehyde (PFA) containing 1% (v/v) Triton-X-100 for 30 minutes at room temperature. Next, cells were trice washed with 400 μl of washing buffer (PBS+/+ with 0.05% (v/v) Tween® 20) and overlayed with 50 μl of primary anti-NP antibody solution (3% (w/v) BSA in PBS+/+) for 2 hours at room temperature. Then, cells were washed tree times with washing buffer followed by incubation with 50 μl of secondary Horse-Radish Peroxidase (HRP) labeled anti-mouse antibody. 1 hour later, cells again were washed with 400 μl of washing buffer and 40 μl 3-Amino-9-ethylcarbazole (AEC)-staining buffer (1×AEC diluted N—N-dimethylformamide in acetate buffer (50 mM ammonium acetate, 8.8 mM H₂02) were added to each well. Following incubation for 30 min at 37° C. until foci could be detected, the staining buffer was removed and cells were washed twice with dH₂O. Air-dried plates were scanned by using the Epson Perfection V500 Photo scan (Epson) at 1200 dpi and total number of foci was determined per well. Since Avicel-medium has a high viscosity that prevents diffusion of virus particles in surrounding media, virus can spread only from one cell to other forming foci.

The viral titer per 1 ml was determined by formula:

Number of foci per well×1000 μl/50 μl×dilution factor⁻¹=ffu/ml, where ffu is foci forming unit.

Preparation of Lung Homogenate for Virus Titration

For analysis of the virus titer in infected lung epithelial cells by foci assay, mice were sacrificed by exsanguination. The pulmonary circulation was flushed with sterile PBS −/− via the right ventricle. Flushed blanched lungs were removed and washed with cold PBS −/−. Lobes were sheared with scissors and remaining tissue was dissociated by pipetting in 1 ml PBS −/− to single cell suspensions. Cells were pelleted by centrifugation at 400×g for 10 min at 4° C. and supernatant was subjected to foci assay as described earlier.

### Example 6—Fixation of Cells for Immunofluorescence Assay

For immunofluorescence assay cells were washed ones with PBS+/+ and fixed with or without extra permeabilization at the indicated time points. Depending on the primary antibodies used cells were either fixed and permeabilzed with organic solvents or fixed with the cross-linking reagent paraformaldehyde. As organic solvents either pre-cooled (−20° C.) 1:1 (v/v) aceton:methanol solution (for NKA α1 staining) or pre-cooled (−20° C.) 100% methanol (for tubulin staining) was used for 3 min at −20° C. followed by three times washing with washing buffer (PBS+/+ with 0.05% (v/v) Tween® 20) and blocked with blocking buffer (bovine serum albumin (BSA) 3% (w/v) in 1×PBS+/+) for one hour at RT or overnight at 4° C. As a cross-linking reagent 4% (w/v) PFA solution was used to fix the cells for 10 min at RT, followed by washing thrice with PBS+/+ containing 30 mM glycine (G-PBS) and subsequently permeabilized with 0.25% (v/w) Triton X-100 for 7 min. Then cells were washed three times with G-PBS and were overlayed with blocking solution (3% (w/v) BSA in G-PBS, G-PBS/BSA) for 30 min at RT. Fixed cells were then treated with 0.25% (v/w) Triton X-100 in G-PBS for 15 minutes, washed with G-PBS three times followed by blocking in G-PBS/BSA for 30 min.

### Example 7—Antibody Staining and Confocal Laser-Scanning Microscopy of Apical NKAα1 Localization in Infected and Non-Infected Calu3 Cells (FIG. 1)

Highly polarized monolayers of Calu3 cells grown on Transwell® inserts at air/liquid-interphase were either left un-infected or were infected with influenza virus A/Puerto Rico/8/34 (H1 N1) at an MOI=5. 20 h p.i.. Infected cells were either left untreated or treated with the ROCK inhibitor (XIII). For immunofluorescence assay cells were washed ones with PBS+/+. Cells were fixed and permeabilzed with pre-cooled (−20° C.) 1:1 (v/v) aceton:methanol solution (for NKAα1 staining) for 3 min at −20° C. followed by three times washing with washing buffer (bovine serum albumin 0.3% (w/v) in 1×PBS+/+) and blocked with blocking buffer (bovine serum albumin 3% (w/v) in 1×PBS+/+) for one hour at RT or overnight at 4° C. For antibody staining cells were then incubated with specific primary antibody (rabbit anti-NP, Thermo-Fisher (PA5-32242): 1:2000; mouse anti-α1 NKA, Millipore/Sigma Aldrich (#05-369): 1:1000) in antibody diluting solution (bovine serum albumin 2% (w/v) in 1×PBS+/+). The antibody dilution was added to the fixed and permeabilized cells for 2 h at RT, followed by washing twice with PBS+/+. The cells were then incubated for 1 h with secondary antibody (chicken anti-rabbit Alexa Fluor 488 and chicken anti-mouse Alexa Fluor 647) diluted in antibody diluting solution (1:1000). Then cells were washed thrice with PBS −/−, once with ddH₂O and cover slips or polyester membrane from Transwells® were mounted on a glass slide with ProLong™ Gold antifade mountant with DAPI (conc. not given by the manufacture) overnight. NKAα1 localization was assessed by an indirect immunofluorescence analysis and subsequent 3D-modeling using Imaris® software. Signals were visualized by using a Leica TCS-SP5 confocal laser-scanning microscope with HCX PL Apo 63×/1.30 GLYC objective and a pinhole—1 airy unit (AU). Z-Stack was acquired using 0.25 μm step size and results were analyzed by Imaris software (Bitplane) (FIGS. 1 and 2).

### Example 8—Analysis of Protein Cell Surface Expression

On-Cell-Western Blot Assay

For the On-Cell Western blot assay, three sets of Calu3 cells were seeded in 96-well with optically clear flat bottom plates at a concentration of 6×10⁴ cells/well. 24 hours later when the cell monolayer was 95% confluent, cells were infected with influenza virus A/Puerto Rico78/34 (H1N1) at an MOI of 2. After 45 min of incubation at 37° C. the inoculum was replaced by the Infection medium (MEM containing 1% Sodium Pyruvate (100×), 1% Non-Essential Amino Acids (100×), 0.5% BSA (30%)) (+/−) inhibitor (Fasudil HCl, Selleckchem: 10 μM; Rho kinase inhibitor RKI-1447 (XIII), Millipore: 5 μM) or solvent of the inhibitor as a control. 24 hours later medium containing either inhibitor or solvent was removed and primary antibodies that recognize an extracellular epitope of HA, M2 or the Na⁺,K⁺-ATPase β1 subunit diluted in PBS+/+(goat polyclonal anti-influenza A virus, Abcam (#ab20841): 1:2000; mouse mono-clonal anti-Influenza A virus M2, ThermoScintific/Invitrogen/Gibco (#MA1-082): 1:1000; mouse mono-clonal anti-P1 NKA, ThermoScintific/Invitrogen/Gibco (#MA3-930): 1:1000) were added to either one set of Calu3 cells and plates were further incubated for 1.5 h at 37° C. at 5% CO₂. Cells were then washed three times with PBS+/+, fixed with 4% PFA for 20 min at RT followed by washing thrice with PBS+/+ for 5 min each. Then, cells were treated with blocking buffer for 45 min at RT and then incubated in dark with the secondary IRDye 800-conjugated anti-mouse, or goat antibody (LI-COR, accordingly to the host of primary antibody) diluted in blocking buffer containing 5 μM DRAQ5™ (a far-red DNA stain) for 1 h at RT. Cells were then washed three times with TBS-T and scanned on the LI-Cor Odyssay Infrared Imager (100 μm resolution, 0.5 mm focus offset). Data were analyzed using Image Studio (LI-COR), Excel (Microsoft) and GraphPad Prism 5 (Graphpad Software, Inc.) software (FIG. 11).

### Example 9—Animal In Vivo Experiment

All animal experiments were performed according to the latest guidelines of the “Federation of European Laboratory Animal Science Associations (FELASA)” and approved by the local committee of the Max-Planck Laboratory for Heart & Lung Research Instituto de Investigación en Biomedicina de Buenos Aires (IBioBA). Six-week-old BALB/c mice (n=5 per group) were infected by intra-tracheal inoculation of 500 plaque-forming units (pfu)/mouse of PR8 in a volume of 30 μl. Fasudil HCl was diluted in sterile PBS −/− and was daily applied intraperitoneally (IP) at a concentration of 10 mg/kg 24 h p.i. during the next 7 days. As a control IP injection of sterile PBS −/− were applied. Body weight was monitored every day until day 8 p.i.. On the day 7 after treatment start (=day 8 p.i.) mice were sacrificed by an overdose of isoflurane.

Wet-to-Dry Lung Weight Ratio

The lung wet-to-dry (W/D) weight ratio was used to analyze lung water accumulation after IAV infection. The animals were sacrificed, dissected, and the lung ‘wet’ weight was measured immediately after its excision. The lungs were then dried in an oven at 60° C. for 5 days and re-weighed as dry weight. The W/D weight ratio was calculated by dividing the wet by the dry weight.

Preparation of Lungs for Histologic Processing

The animals were sacrificed, lungs were perfused via the right ventricle with PBS −/−, removed from chest cavity, fixed in 4% PFA for 24 h and were then embedded in Paraffin (Leica ASP200S). Paraffin embedded lungs were cut into thin sections (3.5 μm) using a Microtome RM2125 (Leica). Slices were mounted on to charged slides and dried overnight at 37° C. Next day, lung sections were stained by Hematoxilin/Eosin by following procedure:

| Step | Time | |
|---|---|---|
| Xylene | 5 | min |
| Xylene | 5 | min |
| 100% ethanol | 30 | s |
| 100% ethanol | 30 | s |
| 100% ethanol | 30 | s |
| 96% ethanol | 30 | s |
| 96% ethanol | 30 | s |
| 70% ethanol | 30 | s |
| 70% ethanol | 30 | s |
| Hematoxylin | 3-5 | min |
| 0.1% HCL | 2 | s |
| Flowing tap water | 5 | min |
| 0.5% Eosin G solution | 3 | min |
| Tap water | 30 | s |
| 70% ethanol | 30 | s |
| 96% ethanol | 30 | s |
| 100% ethanol | 30 | s |
| 100% ethanol | 30 | s |
| 100% ethanol | 30 | s |
| Xylene | 5 | min |
| Xylene | 5 | min |

All microscopic analysis described herein was performed by EVOS FL Auto Cell Imaging System. A total amount of cells in histological cuts was quantified by Aperio CS2 Scanner (Leica Biosystems Imaging Inc., CA, USA) using “Aperio v9 nuclear count algorithm” software (Leica Biosystems Imaging Inc., CA, USA) in collaboration group of Univ.-Prof. Dr. Achim Gruber (Freie Universitst Berlin).

Statistics

Statistical analysis was performed by GraphPad Prism 5 software. The data are given as a mean+either standard error of mean (SEM) or standard deviation of the mean (SD) (indicated in figure legend). The statistical significance of two groups was tested by a two-tailed unpaired Student's t test. The statistical significance of three or more groups were analyzed by one-way ANOVA followed by Tukey's post hoc test. A p value was considered as a significant, if it was less than 0.05, *p<0.05; **p<0.01; ***p<0.005

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### LIST OF REFERENCES

Altschul, Nucl. Acids Res. 25 (1997), 3389-3402
Altschul, J. Mol. Evol. 36 (1993), 290-300
Altschul, J. Mol. Biol. 215 (1990), 403-410
Amano et al. (2010) “Rho-Kinase/ROCK: A Key Regulator of the Cytoskeleton and Cell Polarity” Cytoskeleton (Hoboken). 2010 September; 67(9): 545-554
Assaad et al. (2018) “Assessment of Pulmonary Edema: Principles and Practice.” Journal of Cardiothoracic and Vascular Anesthesia, 32(2), 901-914
Brutlag Comp. App. Biosci. 6 (1990), 237-245
Crystal et al. (2008) “Airway epithelial cells” Proc Am Thorac Soc, vol. 5, pp. 772-777
Feng et al. (2015) “Rho Kinase (ROCK) Inhibitors and Their Therapeutic Potential’ including the information about clinical trials” J. Med. Chem. 2016, 59, 6, 2269-2300
Henikoff Proc. Natl. Acad. Sci., USA, 89, (1989), 10915
Henikoff and Henikoff (1992) ‘Amino acid substitution matrices from protein blocks.’ Proc Natl Acad Sci USA. 1992 Nov. 15; 89(22):10915-9
Johnson and Koval (2009) “Cross-Talk Between Pulmonary Injury, Oxidant Stress, and Gap Junctional Communication.” Antioxidans and Redox Signaling, vol. 11, number 2, pp. 356-367
Liao et al. (2007) “Rho Kinase (ROCK) Inhibitors” J. Cardiovasc Pharmacol. 50(1):17-24
Marconett et al. (2016) “Cross-Species Transcriptome Profiling Identifies New Alveolar Epithelial Type I Cell-Specific Genes” AJRCMB, vol 56, no. 3
Matthay et al. (2019) “Acute Respiratiory distress Syndrome” Nat Rev Dis Primers, 5(1): 18, p. 1-52
McElroy and Kasper (2004) “The use of alveolar epithelial type I cell-selective markers to investigate lung injury and repair” European Respiratory Journal 24: 664-673
McIntyre et al. (1992) “A noninvasive method of predicting pulmonary-capillary wedge pressure” N Engl J Med. 327(24): 1715-20
Mutlu et al. (2005) “Mechanisms of pulmonary edema clearance” Am J Physiol Lung Cell Mol Physiol. 289: L685-695
Ňamendys-Silva et al. (2011) “Acute respiratory distress syndrome caused by influenza B virus infection in a patient with diffuse large B-cell lymphoma” Case Rep Med. 2011; 2011: 647528
Peteranderl et al. (2019) “Influenza A virus infection induces apical redistribution of Na⁺, K⁺-ATPase in lung epithelial cells in vitro and in vivo” American Journal of Respiratory Cell and Molecular Biology Volume 61 Number 3, pp. 395-397
Rackley et al. (2012) “Building and maintaining the epithelium of the lung” The Journal+ of Clinical investigation, vol. 122, no. 8, pp. 2724-2730
Saito N, et al. (1999) “H-series protein kinase inhibitors and potential clinical applications” Pharmacol Ther, 82(2-3), 123-131
Thompson Nucl. Acids Res. 2 (1994), 4673-4680
WO 2009/158587
WO 2012/015760
Wu and Mlodzik (2009). “A quest for the mechanism regulating global planar cell polarity of tissues”. Trends in Cell Biology. 19 (7): 295-305

1. ROCK inhibitor for use in the treatment or prevention of pulmonary edema by
i) preventing apical sodium-potassium-ATPase (NKA) localisation in lung epithelial cells, or
ii) reducing apical NKA localisation in lung epithelial cells compared to the apical NKA localisation before the administration of the ROCK inhibitor,
wherein the pulmonary edema is associated with a virus infection, and
wherein the virus is an influenza virus.

2. ROCK inhibitor for use of claim 1, wherein the ROCK inhibitor is a ROCK 1 or ROCK 1/2 inhibitor.

3. RPCK inhibitor for the use of claim 1, wherein the ROCK inhibitor is selected from the group consisting of Fasudil, Rho XIII, Y27632, Hydroxyfasudil, H-1152-P, Y27632, Y30141, Y32885, Y39983, DW1865, SLx-2119, SR8046, SR6246, Ripasudil, AS1892892, AR12141, AR12432, INS-117548, INS-115644, AT13148, RK11447, SAR407899, Netarsudil, AR12286, PG286*, PG324**, ATS907, AMA0076, Thiazovivin, Azabenzimidazole-aminofurazans, H-0104 Dihydrochloride, DE-104, Olefins, Isoquinolines, Indazoles, pyridinealkene derivatives, H-1152 dichloride, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, quinazoline, AR13154 and/or AMA0428 or combinations thereof.

4. ROCK inhibitor for use of any one of the preceding claims, wherein the pulmonary edema is diagnosed by chest X ray or computer tomography.

5. ROCK inhibitor for use of any one of the preceding claims, wherein the ROCK inhibitor is administered to a subject infected or at risk of infection with an influenza A or influenza B virus.

6. ROCK inhibitor for use of any one of the preceding claims, wherein the lung epithelial cell is an alveolar epithelial cell and/or a bronchial epithelial cell.

7. ROCK inhibitor for use of any one of the preceding claims, wherein the alveolar epithelial cell is a type I or type II alveolar epithelia cell.

8. ROCK inhibitor for use of any one of the preceding claims, wherein the bronchial epithelial cell is a ciliated and/or non-ciliated bronchial epithelia cell.

9. ROCK inhibitor for use according to any one of the preceding claims, wherein the ROCK inhibitor reduces apical NKA localisation in lung epithelial cells when contacting it with an in vitro test system, wherein the test system comprises cultured lung epithelial cells infected with an influenza virus, when compared to apical NKA localisation in lung epithelial cells in the in vitro test system before the contacting.

10. ROCK inhibitor for use according to any one of the preceding claims, wherein the ROCK inhibitor reduces the virus load compared to the virus load before the administration of the ROCK inhibitor.

11. ROCK inhibitor for use according to any one of the preceding claims, wherein the ROCK inhibitor reduces the fluid weight of the lung compared to the fluid weight of the lung present before the administration of the ROCK inhibitor.

12. ROCK inhibitor for use according to any one of the preceding claims, wherein the ROCK inhibitor reduces the infiltration of macrophages into the lung compared to the infiltration of macrophages into the lung before the administration of the ROCK inhibitor.

13. Use of an in vitro test system comprising cultured lung epithelial cells infected with an influenza virus, for the determination of inhibitors effective in preventing or reducing apical NKA localisation in lung epithelial cells.

14. A method for detecting molecules effective in the prophylaxis and/or treatment of a pulmonary edema comprising contacting an in vitro test system comprising cultured lung epithelial cells infected with an influenza virus with a compound of interest, wherein the compound of interest reduces apical NKA localisation in lung epithelial cells, compared to the in vitro test system before the contacting.

15. A test system comprising
i) a ROCK inhibitor;
ii) lung epithelial cells;
iii) an influenza virus; and
iv) means for the detection and cellular localization of NKA.


SEQUENCE LISTING (15 sequences)

SEQ ID NO: 1 - protein, 303 aa, Homo sapiens
MARGKAKEEGSWKKFIWNSEKKEFLGRTGGSWFKILLFYVIFYGCLAGIFIGTIQVMLLTISEFKPTYQDRVAPPGLTQIPQIQKTEISFRPNDPKSYEAYVLNIVRFLEKYKDSAQRDDMIFEDCGDVPSEPKERGDFNHERGERKVCRFKLEWLGNCSGLNDETYGYKEGKPCIIIKLNRVLGFKPKPPKNESLETYPVMKYNPNVLPVQCTGKRDEDKDKVGNVEYFGLGNSPGFPLQYYPYYGKLLQPKYLQPLLAVQFTNLTMDTEIRIECKAYGENIGYSEKDRFQGRFDVKIEVKS

SEQ ID NO: 2 - protein, 290 aa, Homo sapiens
MVIQKEKKSCGQVVEEWKEFVWNPRTHQFMGRTGTSWAFILLFYLVFYGFLTAMFTLTMWVMLQTVSDHTPKYQDRLATPGLMIRPKTENLDVIVNVSDTESWDQHVQKLNKFLEPYNDSIQAQKNDVCRPGRYYEQPDNGVLNYPKRACQFNRTQLGNCSGIGDSTHYGYSTGQPCVFIKMNRVINFYAGANQSMNVTCAGKRDEDAENLGNFVMFPANGNIDLMYFPYYGKKFHVNYTQPLVAVKFLNVTPNVEVNVECRINAANIATDDERDKFAGRVAFKLRINKT

SEQ ID NO: 3 - protein, 279 aa, Homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 4 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 5 - protein, 1020 aa, Homo sapiens
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 6 - protein, 1013 aa, Homo sapiens
MGDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 7 - protein, 1029 aa, Homo sapiens
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 8 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 9 - protein, 354 aa, Homo sapiens
MTALSRSEATEEGGNQQMHRKTASLNSPVSCKEKPDRVEEPPDYSLHWPEGLKGEEIKKCGREGITLNKYNQQYHKLFKDVPLEEVVLKVCSCALQRDFLLQGRLYISPNWLCFHASLFGKDIKVVIPVVSVQMIKKHKMARLLPNGLAITTNTSQKYIFVSLLSRDSVYDLLRRVCTHLQPSSKKSLSVREFSGEPESLEVLIPEMKWRKVCPSSRSLSLPDNIPCIPPSSVDSTDSFFPSRKPPMSEKSRAQVASENGGRWAWPMPGWGPACPKKMPNCSPTAKNAVYEEDELEEEPRSTGELRLWDYRLLKVFFVLICFLVMSSSYLAFRISRLEQQLCSLSWDDPVPGHR

SEQ ID NO: 10 - protein, 432 aa, Homo sapiens
MTELQQDVEDTKPAKVLGKRESKLGSAHSEAENGVEEKKKACRSPTAQSPTPSVEADSPDQKKIISLWSKSSFDGASLASDKNDCKTESKNDPKTERKKSSSSSQYKANMHFHKLFLSVPTEEPLKQSFTCALQKEILYQGKLFVSENWICFHSKVFGKDTKISIPAFSVTLIKKTKTALLVPNALIIATVTDRYIFVSLLSRDSTYKLLKSVCGHLENTSVGNSPNPSSAENSFRADRPSSLPLDFNDEFSDLDGVVQQRRQDMEGYSSSGSQTPESENSRDFHATESQTVLNVSKGEAKPTRADAHVNRVPEGKAKSLPVQGLSETVGILHKVKSQKCPMLHHILIFYAIVVCALIISTFYMRYRINTLEEQLGLLTSIVDTHNTEQAAPSGLRSQVQFNVEVLCQELTANIVKLEKIQNNLQKLLENGD

SEQ ID NO: 11 - protein, 197 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALNRKVHNFQMECSLQAKPAVPTSKLGQAEGRDAGSAPSGGDPAFLGMAVNTLCGEVPLYYI

SEQ ID NO: 12 - protein, 562 aa, Homo sapiens
MAIIYLILLFTAVRGDQICIGYHANNSTEKVDTNLERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKEGSDYPVAKGSYNNTSGEQMLIIWGVHHPIDETEQRTLYQNVGTYVSVGTSTLNKRSTPEIATRPKVNGQGGRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFGNLERRLENLNKRMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 13 - protein, 566 aa, Homo sapiens
MKTIIALSYIFCLALGQDLPGNDNNTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPDSGFFSRLNWLTKSGSTYPVLNVTMPNNDNFDKLYIWGVHHPSTNQEQTSLYVQASGRVTVSTRRSQQTIIPNIGSRPWVRGQSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 14 - protein, 469 aa, Homo sapiens
MNPNQKIITIGSVSLTIATVCFLMQIAILATTVTLHFKQHKCDSPASNQVMPCEPIIIERNITEIVYLNNTTIEKEICPEVVEYRNWSKPQCQITGFAPFSKDNSIRLSAGGDIWVTREPYVSCDPGKCYQFALGQGTTLDNKHSNGTIHDRIPHRTLLMNELGVPFHLGTKQVCVAWSSSSCHDGKAWLHVCVTGDDRNATASFIYDGRLVDSIGSWSQNILRTQESECVCINGTCTVVMTDGSASGRADTRILFIKEGKIVHIGPLSGSAQHIEECSCYPRYPDVRCICRDNWKGSNRPVIDINMEDYSIDSSYVCSGLVGDTPRNDDSSSNSNCRDPNNERGNPGVKGWAFDNGDDVWMGRTISKDLRSGYETFKVIGGWSTPNSKSQVNRQVIVDNNNWSGYSGIFSVEGKSCINRCFYVELIRGRPQETRVWWTSNSIVVFCGTSGTYGTGSWPDGANINFMPI

SEQ ID NO: 15 - protein, 469 aa, Homo sapiens
MNPNQKIITIGSVSLTIATVCFLMQTAILVTTVTLHFKQYECDSPASNQVMPCEPIIIERNITEIVYLNNTTIEKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQEARVWWTSNSIVVFCGTSGTYGTGSWPDGANINFMPI